(19) United States
(12) Patent Application Publication
(10) Pub. No.: US 2023/0149425 A1
(43) Pub. Date: May 18, 2023

(54) PROCESS AND THERAPEUTIC COMPOSITION FOR TREATING AND PREVENTING SEVERE INJECTION SITE REACTIONS

(71) Applicant: Brii Biosciences, Inc., Durham, NC (US)

(72) Inventors: Zhi HONG, Durham, NC (US); Ji MA, Foster City, CA (US); David MARGOLIS, Durham, NC (US); Lianhong XU, Palo Alto, CA (US); Li YAN, Berwyn, PA (US)

(21) Appl. No.: 18/056,761

(22) Filed: Nov. 18, 2022

Related U.S. Application Data
(63) Continuation of application No. 63/398,221, filed on Aug. 15, 2022.
(60) Provisional application No. 63/281,066, filed on Nov. 18, 2021.

(51) Int. Cl. A61K 31/573; A61K 9/00; A61P 25/04

(52) U.S. Cl. CPC A61K 31/573; A61K 9/0019; A61P 25/04

(57) ABSTRACT

Disclosed herein is a method for treating and/or minimize injection site reactions (ISRs) and/or preventing severe ISRs in a subject in need thereof by admixing, co-formulating or co-administering a steroid composition. In some embodiments, this disclosure is directed to a method for treating a central nervous system (CNS) disorder. The method comprises co-administering a steroid composition and a neuroactive steroid (NAS). The method can be used for preventing and/or treating CNS conditions or diseases related to GABA-modulation, such as depression, postpartum depression (PPD) particularly in lactating women, bipolar disorder, dementia, Huntington's disease, Parkinson's disease, and other diseases, and preventing severe ISRs.

### CROSS-REFERENCE

This application claims the benefit of U.S. Provisional Application No. 63/398,221, filed Aug. 15, 2022, and U.S. Provisional Application No. 63/281,066, filed Nov. 18, 2021, each of which is incorporated herein by reference in its entirety.

### FIELD OF THE DISCLOSURE

The present disclosure is directed to processes and therapeutic compositions for treating or preventing severe injection site reaction (ISR) in a subject in need thereof. The present disclosure is further directed to methods and therapeutic compositions for treating a central nervous system (CNS) disorder using a neuroactive steroid (NAS) and a steroid composition.

### BACKGROUND

An injection site reaction (ISR) is a local phenomenon defined as a constellation of symptoms, including swelling, erythema, pruritus, and pain around the site of injection. There are two types of injection site reactions: a local allergic reaction including a flare reaction, and a more severe reaction characterized by damage to the tissue due to extravasation caused by leakage of a small amount of drug into the tissue around the site where it was injected. Symptoms for both reactions typically include redness, tenderness, warmth, and itching, but can include pain, blistering, and severe skin damage. Depending on the cause, injection site reactions may be treated with warm compresses, ice, possibly an antidote for certain drugs that have extravasated, and in extreme cases, plastic surgery.

Injection site reactions (ISRs) can be an issue in the development and clinical use of parenteral drugs. For example, ISR has been found to be a relatively common side effect of injectable TNF-α inhibitors as treatment for rheumatoid arthritis (RA) (Matsui, T. et al., Int'l J. of Medical Sciences, 14(2):102-109, 2017. DOI: 10.7150/ijms.17025). ISR can lead to patient stress, nervousness, pain and a decrease in quality of life (QoL), and may affect adherence to drug regimen in patients. ISR usually occurs within the first month of treatment, lasting for 3 to 5 days. Although most cases of ISR resolve themselves or with cold compresses, severe ISR may require treatment. In some embodiments, topical applications of corticosteroids (Kaiser, C., Rheumatol Intl., 32:295-299, 2012. DOI: 10.1007/s00296-011-2096-3), refrigerated lotions, creams and ointments (Kukowski, B., et al., Patient Preference and Adherence. 15:1091-1100, 2021) and other medications such as antihistamines or acetaminophen can be used to relieve ISR. In some instances, neither systemic nor locally applied corticosteroid treatment prevents development of ISRs (Leonie van Meer, et al., Br J Clin Pharmacol 82:340-351, 2016. DOI:10.1111/bcp.12961).

Side effects such as ISR may become an important factor involved in the patient preference for certain drug selection and treatment adherence. Sometimes, severe ISRs can lead to preclinical and clinical dose limiting toxicities. Currently, the clinical implications and treatment regime for ISRs are not well established.

An important class of parentally administered pharmaceutically active agents that may cause or induce ISRs is neuroactive steroids (NASs). NASs are modulators of y-aminobutyric acid (GABA) receptor complex (GRC) in the central nervous system (CNS). The prime target of NASs is the inhibitory GABA Type A receptors (GABAARs) that attribute to the modulation of neuronal excitability and rapid mood changes. NASs can be produced de novo in the brain from cholesterol or derived from the local metabolism of peripherally derived steroid precursors. The endogenous neurosteroids can include 5α-pregnane-3α-ol-20-one (allopregnanolone, also known as brexanolone) and 5α-pregnane-3α,21-diol-20-one (THDOC) (Majewska Md., et al., Science. 232:1004-7, 1986 [PubMed: 2422758]). Synthetic neuroactive steroids, such as alphaxalone, can also selectively potentiate responses to GABA (Harrison N L, et al., J Physiol (Lond). 346:42, 1984). Many CNS disorders including a variety of behavioral states such as anxiety levels, panic, stress response, seizures, sleep, vigilance and memory, etc., can be related to GABAARs function and can be influenced by NASs and their synthetic derivatives (Zorumski, C F., et al., Neurosci. Biobehavioral Rev. 37:109-122, 2013. DOI: 10.1016/j.neubiorev.2012.10.005).

Given its critical role in the function of neuronal circuits, GABAARs are the target for numerous clinically relevant drugs. Brexanolone (also known as allopregnanolone) and ganaxolone are known positive allosteric modulators of the GABAARs causing a global inhibition of central nervous system (CNS). A brexanolone solution formulation product for intravenous injection, ZULRESSO®, developed by and sold under registered trademark of Sage Therapeutics (Cambridge, Mass., USA), was recently approved by US Food and Drug Administration (FDA, March 2019) for the treatment of postpartum depression (PPD), a serious and potentially life-threatening condition, for which no current pharmacotherapies are specifically indicated. However, ZULRESSO is inconvenient to use and requires the administration to a patient by continuous intravenous (IV) infusion that lasts for a total of about 60 hours (2.5 days). A new oral drug, positive allosteric modulator of GABA_{A}Rs, known as SAGE-217, has shown mixed results in reduction of depressive symptoms in clinical trials after administration of the drug for 14 days. In addition, SAGE-217 exhibited more adverse events than the placebo (Gunduz-Bruce, H., et al., N. Engl. J. Med. 381(10):903-911, September 2019; U.S. Pat. No. 9,512,165 and PCT Publication No.: WO2014/169833). Many new molecules have also been proposed, such as those disclosed in U.S. Pat. No. 9,777,037 and US Patent Publication No.: 20180340005A1. However, their effectiveness in treating human CNS disorders is not clear. In addition, side effects such as skin flushing (redness and warm feeling in skin) can occur with ZULRESSO.

Therefore, a need remains for better compounds for modulating the functions of GABA_{A}Rs for the treatment of CNS disorders and reducing side effects such as ISR.

### SUMMARY

In some aspects, the present disclosure is directed to a process for treating or preventing injection site reaction (ISR) in a subject in need thereof, the process comprising administering to the subject a steroid (e.g., corticosteroid) composition and a pharmaceutical composition comprising an active agent via injection, wherein: (1) the steroid composition and the pharmaceutical composition are co-formulated; (2) the steroid composition is admixed with the pharmaceutical composition; (3) the steroid composition is co-administered with the pharmaceutical composition at a co-administration site at or adjacent to the injection site of the pharmaceutical composition; or a combination thereof, wherein the pharmaceutical composition is administered to the subject via intravenous (IV) injection, intramuscular (IM) injection or subcutaneous injection (SC) at the injection site.

In some embodiments, the ISR is severe ISR.

In some embodiments, the active agent is a neuroactive steroid (NAS) that is a positive modulator of gamma-aminobutyric acid type A (GABA_{A}) receptor.

In some embodiments, the NAS is administered to the subject to treat a central nervous system disorder related to gamma-aminobutyric acid type A (GABA_{A}) receptor modulation. In some embodiments, the CNS disorder is post-partum depression (PPD).

In some embodiments, the steroid composition is a corticosteroid composition.

The present invention is also directed to methods for treating a central nervous system (CNS) disorder related to gamma-aminobutyric acid type A (GABA_{A}) receptor modulation in a subject in need thereof, the method comprising administering a neuroactive steroid (NAS) that is a positive modulator of the GABA_{A} receptor, or a pharmaceutically acceptable salt or composition thereof, and a steroid (e.g., corticosteroid) composition to the subject via injection, wherein the neuroactive steroid (NAS) and the steroid composition are administered to the subject via intramuscular (IM) injection or subcutaneous injection (SC).

In some embodiments, the subject has or is susceptible to an ISR. In some embodiments, the ISR is severe ISR

In some embodiments, the NAS, composition thereof, or pharmaceutically acceptable salt or composition thereof, and steroid composition are administered at at least one injection site.

In some embodiments, the steroid composition is a corticosteroid composition.

The present disclosure is further directed to a therapeutic composition comprising (i) a neuroactive steroid (NAS) that is a positive modulator of gamma-aminobutyric acid type A (GABA_{A}) receptor, a pharmaceutically acceptable salt thereof or composition thereof, (ii) a steroid (e.g., corticosteroid) composition, and (ii) one or more pharmaceutically acceptable excipients.

In some embodiments, the therapeutic composition is formulated for treating a CNS disorder related to gamma-aminobutyric acid type A (GABA_{A}) receptor modulation in a subject.

In some embodiments, the therapeutic composition is for treating or preventing ISR in a subject. In some embodiments, the ISR is severe ISR.

In some embodiments, the steroid composition is a corticosteroid composition.

### BRIEF DESCRIPTION OF THE FIGURES

FIG. 1A-FIG. 1D. Schematic illustrations of exemplary particle size distributions. FIG. 1A: a pharmaceutical composition comprises minimum amounts, such as less than 1% of small particles and mostly large particles. FIG. 1B: A pharmaceutical composition comprises some small particles and mostly large particles. FIG. 1C: A pharmaceutical composition comprises increasing amounts of small particles and large particles. FIG. 1D: A pharmaceutical composition comprises comparable amounts of small particles and large particles. D50=mass-median-diameter (MMD), wherein 50% of particles are below and 50% of particles are above a given diameter. Mean Large=mean particle size of the large particles. Mean Small=mean particle size of the small particles.

FIG. 2A—FIG. 2E. Examples of pharmaceutical compositions comprising brexanolone. FIG. 2A: Brexanolone structure. FIG. 2B: Small brexanolone particle size distribution. FIG. 2C: Large brexanolone particle size distribution. FIG. 2D-FIG. 2E: Pharmacokinetics (PK) in rats showing rat plasma brexanolone concentrations over time after administration. Legend: Open diamond, brexanolone suspension of small particle, 25 mg/kg; Open square, brexanolone suspension of large particles, 25 mg/kg; Solid square, IV solution (Comparative) 1 mg/kg; and Solid triangle, IM solution (Comparative) 12.5 mg/kg.

FIG. 3A—FIG. 3E. Examples of pharmaceutical compositions comprising ganaxolone. FIG. 3A: Ganaxolone structure. FIG. 3B: Distribution of 4.1 ganaxolone particles. FIG. 3C: Distribution of 3.6 μm ganaxolone particles. FIG. 3D-FIG. 3E: Pharmacokinetics (PK) in rats showing rat plasma concentrations over time after administration. Legend: Open diamond, ganaxolone suspension of 1 μm particles, 25 mg/kg; Open square, ganaxolone suspension of 4 μm particles, 25 mg/kg; Solid square, IV solution (Comparative); and Solid diamond, IM solution (Comparative).

FIG. 4A-FIG. 4B. Examples of Powder X-Ray Diffraction (PXRD) diffractograms. FIG. 4A: Pre-processing commercial brexanolone showing one major peak at position 18.15 2θ. FIG. 4B: Crystalline Form A brexanolone showing multiple peaks at 7.25 and 18.15 2θ.

FIG. 5. Plasma brexanolone concentrations after co-administration of BRII-296 and Triamcinolone Kenalog-40 (40 mg/mL) in healthy adults.

FIG. 6A-FIG. 6B. Plasma brexanolone concentrations after co-administration of BRII-296 and selected steroid. FIG. 6A: Plasma brexanolone (BXN) concentrations after co-administration of BRII-296 at 300 and 600 mg and Depo-Medrol 80 at 10-40 mg per injection site in healthy adults. FIG. 6B: Plasma brexanolone (BXN) concentrations after co-administration of BRII-296 at 600 mg and Depo-Medrol 80 at 20-40 mg per injection site in healthy adults.

### DETAILED DESCRIPTION

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As used herein:

The term “γaminobutyric acid type A receptors”, “GABA_{A} receptors”, “GABA_{A}Rs”, “GABA_{A}R”, “GABA_{A}Rs”, “GABA_{A}R” or a grammatic variation thereof, either in singular or in plural form, refers to gamma-aminobutyric acid type A receptors (GABA_{A}Rs) that are a class of receptors that respond to the neurotransmitter gamma-aminobutyric acid (GABA). GABA is the principal inhibitory neurotransmitter in the cerebral cortex that is important for maintaining the inhibitory state that counterbalances neuronal excitation. Disorder in GABA_{A} receptors or imbalance of GABA and neuroexcitation can lead to a wide range of brain circuits and disorders related to GABA function that are central to a variety of behavioral states such as anxiety levels, panic, stress response, seizures, sleep, vigilance and memory.

A number of natural and synthetic neuroactive steroids can bind to GABA_{A}Rs and modulate their activities.

The term “neuroactive steroid”, “NAS”, “neuroactive steroids”, “NASs” or a grammatic variation thereof refers to one or more neurosteroids that exert inhibitory actions on neurotransmission, specifically, on the GABA_{A} receptors and that are modulators of γ-aminobutyric acid (GABA) receptor complex (GRC) in the central nervous system (CNS). Main examples can include, but not limited, tetrahydrodeoxycorticosterone (THDOC), androstane, androstane 3α-androstanediol, cholestane cholesterol, pregnane, pregnane pregnanolone (eltanolone), allopregnanolone, brexanolone, ganaxolone and SAGE-217. The NASs disclosed herein can be identified by names based on the nomenclature recommended by International Union of Pure and Applied Chemistry (IUPAC) or other nomenclature systems. The NASs can also be identified by chemical structure drawings. Unless expressly stated to the contrary in a particular context, the names and the structures may be used interchangeably throughout this disclosure.

The term “injection site reaction”, “ISR”, “injection site reactions”, “ISRs” or a grammatical variation thereof refers to a disorder characterized by an intense adverse reaction (usually immunologic) developing at the site of an injection, such as symptoms including, but not limited to, swelling, erythema, pruritus, pain, blistering, redness, tenderness, warmth, itching, and skin damage around a site of a subject, such as a patient, where a drug is injected (herein “injection site”). An injection site reaction can be inflammation in or damage to the tissue surrounding the injection site and can be a local allergic reaction such as a flare reaction, or a more severe reaction characterized by damage to the tissue due to extravasation caused by a small amount of drug leaking from the blood vessel where it was injected. According to National Cancer Institute Common Terminology Criteria for Adverse Events (NCI CTCAE) (v5.0, November 2017) and U.S. Department of Health and Human Services, Division of AIDS (DAIDS) Table for Grading the Severity of Adult and Pediatric Adverse Events, Corrected Version 2.1 (July 2017), ISR can be categorized at least as: Grade 1-Grade 5. For this disclosure, ISRs at a level of Grade 1 or less can be considered “moderate” or “mild”, and ISRs at a level of Grade 2 and above can be considered as “severe”. Detailed categorization of site reactions to intramuscular injections is shown in Table 2.

The term “steroid composition” refers to a steroid composition that comprises one or more corticosteroids, and optionally one or more pharmaceutically acceptable carriers or excipients. In some embodiments, the corticosteroid comprises alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, triamcinolone hexacetonide, triamcinolone, or a combination thereof.

The term “breastfeeding” or a grammatical variation refers to delivering breast milk of the woman directly to an infant, extracting breast milk from the woman using a device and subsequently delivering to the infant, extracting breast milk from the woman using a device and storing the breast milk for a short period of time and subsequently delivering the stored breast milk to the infant, or a combination thereof. In some embodiments, a subject disclosed herein is a lactating woman who is breastfeeding an infant frequently on a regular basis. In some embodiments, a subject disclosed herein is a lactating woman who is breastfeeding and in need for treatment or prevention of postpartum depression (PPD). In some embodiments, a subject disclosed herein is a lactating woman who is breastfeeding an infant frequently on a regular basis and in need for treatment or prevention of postpartum depression (PPD).

The term “admix”, “ad-mix”, “co-admix”, “admixed”, “co-admixed”, “admixing”, “co-admixing” or a grammatical variation thereof refers to mixing two or more different drug products prior to administration, so only one administration, such as an injection, is performed for the two or more drug products. Admixing can usually be done in a secondary container where the two or more drug products can be mixed together, and can be kept for a limited time (usually less than a few days, such as less than 1-3 day, less than 24 hours, less than 8 hours, or less than 4 hours) prior to administration.

The term “coformulation”, “coformulate”, “coformulating”, “co-formulating”, “co-formulated”, “co-formulate”, “co-formulation”, or a grammatical variation thereof refers to two or more drug substances that are formulated together in the same primary packaging (a pill for an oral dose, for example, a vial for an injectable). There is no admixing necessary because the two or more drug substances are already mixed. The two or more drug substances coformulated together can be stored for an extended period of time, such as 6 months, 12 months, 24 months or longer. One or more pharmaceutical acceptable excipients can be used in co-formulation.

The term “pre-mix” can refer to “admix”, “admixing”, “admixed”, “coformulate”, “coformulated”, “coformulating”, “co-formulate”, “co-formulated”, “co-formulating”, or a combination thereof.

The term “co-packaging”, “co-package”, “co-packaged”, or a grammatical variation thereof refers to two or more individually packaged drug products that are provided together under one single secondary packaging (pack or carton or box); they can be co-administered or not. They can be admixed or administered separately.

The term “coadministration”, “co-administration”, “co-admin”, “coadmin”, “co-administered” or a grammatical variation refers to administration of two or more drugs, each in a separate formulation, to a same subject at same time or within a limited time between the administrations. For oral administration, co-administration can be performed to a subject at the same time or within a limited time between the administrations, such as within a few second to a few minutes. For injection, co-administration can be performed to the same subject at the same time or within a limited time between the administrations, such as within a few seconds to a few minutes or a few hours, at the same injection site or at different injections sites of the subject.

As used herein, the term “treating” with regard to a subject, refers to improving at least one symptom of the subject's disorder. Treating can be curing, improving, or at least partially ameliorating a disorder. The term “therapeutic effect” as used herein refers to a desired or beneficial effect provided by the method and/or the composition. For example, the method for treating a disclosed disease or condition provides a therapeutic effect when the method reduces at least one symptom of the disease or condition in a subject.

As used herein, the term “preventing” with regard to a subject refers to reducing or eliminating the onset of the symptoms or complications of a disease, condition or disorder. In some embodiments, the symptoms or complications are reduced or eliminated in a subject that is predisposed to the disease, condition, or disorder. The patches of the present disclosure are capable of preventing or treating such diseases, condition, or disorders. In some embodiments, a “method of preventing” and “method of prophylaxis treatment” are used interchangeably.

### Methods of the Disclosure

In some embodiments, the present disclosure is directed to a process for treating or preventing injection site reaction (ISR) in a subject in need thereof, the process comprising administering to the subject a steroid composition and a pharmaceutical composition comprising an active agent via injection, wherein: (1) the steroid composition and the pharmaceutical composition are co-formulated; (2) the steroid composition is admixed with the pharmaceutical composition; (3) the steroid composition is co-administered with the pharmaceutical composition at a co-administration site at or adjacent to the injection site of the pharmaceutical composition; or a combination thereof, wherein the pharmaceutical composition is administered to the subject via intravenous (IV) injection, intramuscular (IM) injection or subcutaneous injection (SC) at the injection site.

The injection site reaction referred to throughout this disclosure can be caused, induced, or expected to be caused or induced by the injection of the pharmaceutical composition comprising an active agent at the injection site. In some embodiments, the active agent is a NAS that is a positive modulator of gamma-aminobutyric acid type A (GABA_{A}) receptor. In some embodiments, the injection site reaction is caused by the injection of a pharmaceutical composition comprising a neuroactive steroid (NAS) for treating a central nervous system (CNS) disorder related to gamma-aminobutyric acid type A (GABA_{A}) receptor modulation in a subject in need thereof.

In some embodiments, the process is directed to prophylaxis treatment of injection site reaction caused or induced by the injection of a pharmaceutical composition in a subject. In some embodiments, the process is directed to preventing injection site reaction that is expected to be caused or induced by the injection of a pharmaceutical composition in a subject prior to the ISR appearing in the subject. In some embodiments, the process is directed to prophylaxis treatment of injection site reaction caused or induced by the injection of a neuroactive steroid (NAS) useful for treating a central nervous system (CNS) disorder related to gamma-aminobutyric acid type A (GABA_{A}) receptor modulation in a subject in need thereof.

In some embodiments, the steroid composition is admixed with the pharmaceutical composition prior to injection and administered together at an injection site. The steroid composition and the pharmaceutical composition can also be pre-mixed together to form a therapeutic composition, and administered to a subject in need thereof via one or more injections at one or more injection sites.

In some embodiments, the steroid composition is co-formulated with the pharmaceutical composition and administered together at the injection site. In some embodiments, the steroid composition is co-formulated with the pharmaceutical composition to form a therapeutic composition as disclosed herein. The steroid composition and the pharmaceutical composition can be co-formulated with one or more pharmaceutical acceptable excipients disclosed herein. The steroid composition co-formulated with the pharmaceutical composition can be administered to a subject in need thereof via one or more injections at one or more injection sites.

In some embodiments, the therapeutic composition comprises any pharmaceutical composition disclosed herein.

In some embodiments, the steroid composition is co-administered to the subject within 0 to 10 days from administration of the pharmaceutical composition to the subject, e.g., 0 days, 1 day, 2 days, 3 days, 4 days, 5 days, 6 days, 7 days, 8 days, 9 days, or 10 days, inclusive of all values and subranges therebetween. The steroid composition can be co-administered to the subject before or after the ISR appears in the subject. In some embodiments, the steroid composition is co-administered to the subject within 0 to 60 minutes from administration of the pharmaceutical composition to the subject, e.g., 0 minutes, 5 minutes, 10 minutes, 15 minutes, 20 minutes, 25 minutes, 30 minutes, 35 minutes, 40 minutes, 45 minutes, 50 minutes, 55 minutes, or 60 minutes, inclusive of all values and subranges therebetween. In some embodiments, the pharmaceutical composition and the steroid composition are administered simultaneously, i.e., administered within 0 to a few seconds of each other. In some embodiments, the steroid composition is administered to the subject within 10 seconds to 60 minutes, 1 to 60 minutes, 5 to 60 minutes, 10 to 60 minutes, 15 to 60 minutes, 20 to 60 minutes, or 40 to 60 minutes from the pharmaceutical composition is administered to the subject. In some embodiments, the steroid composition is co-administered to the subject within 0 to 60 minutes before or after the pharmaceutical composition is administered to the subject.

In some embodiments, the steroid composition is administered to the subject at a co-administration site that is within 0 to 10 centimeters from the injection site of the pharmaceutical composition, e.g., 0 centimeters, 1 centimeter, 2 centimeters, 3 centimeters, 4 centimeters, 5 centimeters, 6 centimeters, 7 centimeters, 8 centimeters, 9 centimeters, or 10 centimeters, inclusive of all values and subranges therebetween. In some embodiments, the steroid composition is administered to the subject at the co-administration site that is within 0 to 5 centimeters, 0 to 4 centimeters, 0 to 3 centimeters, 0 to 2 centimeters, or 0 to 1 centimeter, from the injection site of the pharmaceutical composition.

In some embodiments, the steroid composition is co-administered to the subject via intramuscular (IM) injection or subcutaneous injection (SC). In some embodiments, the steroid composition is co-administered to the subject via IM injection. In some embodiments, the steroid composition is co-administered to the subject via SC injection.

In some embodiments, the steroid composition, the pharmaceutical composition, or both the steroid composition and the pharmaceutical composition, are administered to the subject in a therapeutic dose or a sub-therapeutic dose. In some embodiments, the steroid composition and the pharmaceutical composition each administered to the subject in a therapeutic dose. In some embodiments, the steroid composition and the pharmaceutical composition is each administered to the subject in a sub-therapeutic dose. In some embodiments, the steroid composition is administered to the subject in a therapeutic dose and the pharmaceutical composition is administered to the subject in a sub-therapeutic dose. In some embodiments, the steroid composition is administered to the subject in a sub-therapeutic dose and the pharmaceutical composition is administered to the subject in a therapeutic dose. In one embodiment, the steroid composition is administered to the subject in a therapeutic dosage or a sub-therapeutic dosage. In another example, the pharmaceutical composition is administered to the subject in a therapeutic dosage or a sub-therapeutic dosage. In yet another example, both the steroid composition and the pharmaceutical composition are each administered to the subject in a respective therapeutic dose or a respective sub-therapeutic dose.

In some embodiments, the steroid composition is a corticosteroid composition. In some embodiments, the corticosteroid composition comprises a corticosteroid selected from the group consisting of alclometasone dipropionate, amcinonide, beclometasone, betamethasone (such as CELESTONE® SOLUSPAN®, registered trademarks of Schering Corporation), betamethasone dipropionate, betamethasone valerate, budesonide, ciclesonide, clobetasol propionate, clobetasone butyrate, cortisone, cortisone acetate, desonide, dexamethasone, dexamethasone acetate (such as those available under the brand name DECADRON-LA), dexamethasone sodium (such as those available under DECADRON®, trademark of PRAGMA PHARMACEUTICALS, or SOLUREX®, trademark of Alexion Pharmaceuticals, Inc.), fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate (such as those available under DEPO-MEDROL®, a trademark of Pfizer Inc.), mometasone, mometasone furoate, prednicarbate, prednisolone, prednisone, tixocortol pivalate, triamcinolone acetonide, triamcinolone hexacetonide (such as those available under ARISTOSPAN®, a trademark of SANDOZ CANADA INC.), triamcinolone acetonide (such as those available under the brand name KENALOG), and a combination thereof. In some embodiments, the corticosteroid is selected from the group consisting of methylprednisolone acetate (such as those available under DEPO-MEDROL®), triamcinolone acetonide (KENALOG), triamcinolone hexacetonide (such as those available under ARISTOSPAN®), dexamethasone acetate (such as those available under the brand name DECADRON-LA), dexamethasone sodium (such as those available under trademarks DECADRON® or SOLUREX®), and a combination thereof. As used throughout this disclosure, trademarks or brand names of certain corticosteroids are used as non-limiting examples only. In some embodiments, the corticosteroid composition comprises methylprednisolone.

In some embodiments, the steroid composition is administered to the subject in a single dose ranging from 0.01 to 50 mg of the steroid per kilogram of body weight of the subject. In some embodiments, the steroid composition is administered to the subject in a single dose ranging from 0.01 to 1 mg, 0.01 to 2 mg, 0.01 to 4 mg, 0.01 to 5 mg, 0.01 to 8 mg, 0.01 to 10 mg, 0.01 to 20 mg, 0.01 to 30 mg, 0.01 to 40 mg and 0.01 to 50 mg of the steroid per kilogram of body weight of the subject.

In some embodiments, the single steroid dose comprises about 1 mg to about 450 mg of the steroid, e.g., about 1 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, or about 450 mg, inclusive of all values and subranges therebetween. The single steroid dose is packaged in a steroid packing unit. Each steroid packing unit can be packaged in a vial, an ampoule, a syringe, a tube, or any packaging format that is suitable for injections. One or more steroid packing units is used for an injection. As defined herein, the term “about 1 mg to about 450 mg of the steroid composition” refers to about 1 mg to about 450 mg of the steroid, such as a corticosteroid. In certain embodiments, the single steroid dose comprises about 1 mg to about 450 mg of a corticosteroid is packaged in a vial. In certain embodiments, the single steroid dose comprises about 1 mg to about 450 mg of Depo-Medrol is packaged in a vial.

In some embodiments, the steroid composition is administered to a subject in a single site dose comprising from 10 to 100 mg of a steroid (e.g., a corticosteroid) for each of the injection site and the pharmaceutical composition is administered to the subject in a single site pharmaceutical composition dose comprising from 30 mg to 1200 mg an active agent (e.g., a NAS) for each of the injection sites. The single site dose of the steroid composition can comprise 10 to 100 mg of the steroid in one embodiment, 20 to 100 mg in another embodiment, 30 to 100 mg in yet another embodiment, 40 to 100 mg in yet another embodiment, 50 to 100 mg in yet another embodiment, 60 to 100 mg in yet another embodiment, 70 to 100 mg in yet another embodiment, 80 to 100 mg in yet another embodiment, or 90 to 100 mg in yet another embodiment, per injection site. In some embodiments, the single site dose of the steroid composition comprises 30 to 40 mg, 40 mg, 40 to 50 mg, 50 mg, 50 to 60 mg, 60 mg, 60 to 70 mg, 70 mg, 70 to 80 mg, 80 mg, 80 to 90 mg, 90 mg, or 90 to 100 mg of the steroid per injection site. In some embodiments, the single site dose of the pharmaceutical composition comprises from 30 mg to 1200 mg in one embodiment, 40 mg to 1200 mg in another embodiment, 50 to 1200 mg in yet another embodiment, 60 to 1200 mg in yet another embodiment, 70 to 1200 mg in yet another embodiment, 80 to 1200 in yet another embodiment, 90 to 1200 mg in yet another embodiment, 100 to 1200 in yet another embodiment, 200 to 1200 mg in yet another embodiment, 300 to 1200 mg in yet another embodiment, 400 to 1200 mg in yet another embodiment, 500 to 1200 mg in yet another embodiment, 600 to 1200 in yet another embodiment, 700 to 1200 mg in yet another embodiment, 800 to 1200 mg in yet another embodiment, 900 to 1200 mg in yet another embodiment, 1000 to 1200 mg in yet another embodiment, or 1100 to 1200 mg in yet another embodiment of the active agent (e.g., NAS), per injection site. In some embodiments, the single site dose of the pharmaceutical composition comprises from 100 to 800 mg, 200 to 800 mg, 300 to 800 mg, 400 to 800 mg, 500 to 800 mg, and 600 to 800 mg of the active agent (e.g., NAS) per injection site. In some embodiments, the single site dose of the pharmaceutical composition comprises 300 mg of the active agent (e.g., NAS) per injection site. All ranges cited herein are inclusive. A combination of any of the aforementioned single site steroid dose and the single site pharmaceutical composition dose can be suitable. In some embodiments, a pre-mixed therapeutic composition comprises 10 to 100 mg of the steroid and 50 to 1200 mg of the active agent. In some embodiments, a pre-mixed therapeutic composition comprises 20 to 40 mg of the steroid and 200 to 800 mg of the active agent. The single site steroid dose can be administered using one or more dose units and the single site pharmaceutical composition dose can be administered using one or more dose units. In some embodiments, a single site dose of the steroid composition and a single site dose of the pharmaceutical composition are administered using 2 injections each comprising 20 mg of the steroid and 300 mg of the active agent.

In some embodiments, the steroid composition and the pharmaceutical composition are formulated in an injection volume in a range of from 0.5 mL to 6 mL for each injection site. In some embodiments, the steroid composition and the pharmaceutical composition are formulated in an injection volume in a range of from 0.5 mL to 1 mL in one embodiment, 0.5 mL to 2 mL in another embodiment, 0.5 mL to 3 mL in yet another embodiment, 0.5 mL to 4 mL in yet another embodiment, 0.5 mL to 5 mL in yet another embodiment, 0.5 mL to 6 mL in yet embodiment, 1 mL to 2 mL in yet another embodiment, 1 mL to 3 mL in yet another embodiment, 1 mL to 4 mL in yet another embodiment, 1 mL to 5 mL in yet another embodiment, 1 mL to 6 mL in yet another embodiment, 2 mL to 3 mL in yet another embodiment, 2 mL to 4 mL in yet another embodiment, 2 mL to 5 mL in yet another embodiment, 2 mL to 6 mL in yet another embodiment, 3 mL to 4 mL in yet another embodiment, 3 mL to 5 mL in yet another embodiment, and 3 mL to 6 mL in yet another embodiment, for each injection site. The steroid composition and the pharmaceutical composition in the injection volume can be administered using one or more units or sub-volumes. In some embodiments, a 6 mL injection volume is administered using 2 injections, each having a volume of 3 mL.

In some embodiments, the ISR is caused or induced by a pharmaceutical composition selected from a vaccine, a nucleic acid vaccine, an mRNA vaccine, an attenuated vaccine, or a viral vector vaccine. In some embodiments, the ISR is caused or induced by a pharmaceutical composition comprising an antibiotic, a chemotherapy drug, a neuroactive steroid, a tumor necrosis factor-α (TNF-α) inhibitor, a biologic drug, or an antibody drug. In some embodiments, the antibody drug is adalimumab, ixekizumab, secukinumab, or ustekinumab. In some embodiments, the chemotherapy drug is bleomycin, CABENUVA (cabotegravir and rilpivirine extended-release injectable suspension), cabotegravir, carboplatin, carmustine (BCNU®), cisplatin, cladribine (LEUSTATIN®), dacarbazine (DTIC-DOME®), daunorubicin (CERUBIDINE®), denileukin diftitox, dolasetrone (ANZEMEt®), doxorubicin (ADRIAMYCIN®, RUBEX®), doxorubicin liposome, epirubicin (ELLENCE®), etoposide, fulvestrant (FASLODEX®), idarubicin (IDAMYCIN®), ifosfamide, interferon (IFN) beta, mechlorethamine (MUSTARGEN®), mitomycin (MUTAMYCIN®), mitoxantrone (NOVANTRONE®), paclitaxel (TAXOL®, ONXAL™), pamidronate (AREDIA®), pegfilgrastim (NEULASTA®), plicamycin (MITHRACIN®), rilpivirine, streptozocin (ZANOSAR®), teniposide (VUMON®), thiotepa, vinblastine (VELBAN®), vincristine (ONCOVIN®, VINCASAR®), vinorelbine (NAVELBINE®), a neuroactive steroid (NAS), or a combination thereof. In some embodiments, the biologic drug is EMGALITY (galcanezumab-gnlm), In some embodiments, the TNF-α inhibitor is etanercept. In some embodiments, the NAS is brexanolone, ganaxolone, zuranolone, a prodrug thereof, or a combination thereof.

In some embodiments, the ISR is caused or induced by injection of a neuroactive steroid (NAS) that is a positive modulator of gamma-aminobutyric acid type A (GABA_{A}) receptor, or a pharmaceutically acceptable salt thereof. The NAS can be administered to the subject to treat a central nervous system (CNS) disorder related to gamma-aminobutyric acid (GABA) A receptor modulation. In some embodiments, the CNS disorder is PPD.

In some embodiments, the pharmaceutical composition comprises a neuroactive steroid (NAS) that is a positive modulator of gamma-aminobutyric acid type A (GABA_{A}) receptor, or a pharmaceutically acceptable salt thereof. In some embodiments, the disease is a central nervous system (CNS) disorder related to gamma-aminobutyric acid (GABA) A receptor modulation, wherein the CNS disorder is selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

In some embodiments, the pharmaceutical composition comprises a neuroactive steroid (NAS) that is a positive modulator of gamma-aminobutyric acid type A (GABA_{A}) receptor. In some embodiments, the neuroactive steroid (NAS) composition comprises brexanolone, long-acting brexanolone, slow-releasing brexanolone, ganaxolone, zuranolone, a prodrug thereof, or a combination thereof. In some embodiments, the neuroactive steroid (NAS) is selected from the group consisting of brexanolone, long-acting brexanolone, slow-releasing brexanolone, ganaxolone, zuranolone, a prodrug thereof, and a combination thereof. In some embodiments, the pharmaceutical composition comprising an NAS is the composition provided in Table 1.

In certain embodiments, the NAS comprises a prodrug of brexanolone, ganaxolone, zuranolone, or a combination thereof. In certain embodiments, the NAS comprises a prodrug of brexanolone. In certain embodiments, the NAS comprises a prodrug of ganaxolone. In certain embodiments, the NAS comprises a prodrug of zuranolone, In some embodiments the NAS comprises a prodrug NAS as described, for instance, in WO 2021/174205 A1, pages 15-36, individually or in a combination, which is incorporated herein for all purposes. In certain embodiments, the NAS comprises (3R,5S,8R,9S,10S,13S,14S,17S)-17-acetyl-3,10,13-trimethylhexadecahydro-1H-cyclopenta[a]phenanthren-3-yl ((5-methyl-2-oxo-1,3-dioxol-4-yl)methyl) carbonate. In some embodiments, the NAS comprises [(3R,5S,8R,9S,10S,13S,14S,17S)-17-acetyl-10,13-dimethyl-2,3,4,5,6,7,8,9,11,12,14,15,16,17-tetradecahydro-1H-cyclopenta[a]phenanthren-3-yl](5-methyl-2-oxo-1,3-dioxol-4-yl)methyl carbonate.

In some embodiments, the neuroactive steroid (NAS) comprises a brexanolone crystalline form.

In some embodiments, the NAS composition comprises brexanolone aqueous suspension BRII-296, a long-acting brexanolone.

In some embodiments, the neuroactive steroid (NAS) is administered to the subject in a single dose comprising about 0.05 to 50 mg of NAS per kilogram of body weight of the subject. In some embodiments, the single dose of the neuroactive steroid comprises about 0.05 to 50 mg, 0.1 to 50 mg, 0.5 to 50 mg, 1 to 50 mg, 5 to 50 mg, 10 to 50 mg, 15 to 50 mg, 20 to 50 mg, 25 to 50 mg, 30 to 50 mg, 40 to 50 mg, 0.05 to 40 mg, 0.05 to 30 mg, 0.05 to 20 mg, 0.05 to 10 mg, 0.05 to 8 mg 0.05 to 5 mg, or 0.05 to 1 mg of NAS per kilogram of body weight of the subject. For any of the NAS prodrugs, the amount of each NAS prodrug dose can be converted to a respective single dose equivalent based on molecular weights and biological efficacy.

The body weight used herein throughout the disclosure refers to the body weight of the subject, unless otherwise specified.

The neuroactive steroid (NAS) can be formulated and packaged for single injection at a single dose. In some embodiments, the single dose is about 50 mg to 1,000 mg of the neuroactive steroid. In some embodiments, the single dose ranges from about 50 mg to about 600 mg of the neuroactive steroid. The single dose can be packaged in an NAS packing unit. In some embodiments, a single dose is packaged in a vial, an ampoule, a tube or a pre-filled syringe. One or more NAS packing units can be used for an injection.

Suitable to the process of this disclosure, the neuroactive steroid can be formulated to reach a maximum plasma concentration (Cₘₐₓ) in about 30 minutes to 6 hours and maintains a plasma concentration of more than about 5% of the Cₘₐₓ for at least about 5 days, in the subject, after a single dose of the pharmaceutical composition comprising the neuroactive steroid (NAS).

In some embodiments, the disclosed pharmaceutical composition comprises a neuroactive steroid in a concentration of about 5 mg/mL to about 800 mg/mL. In some embodiments, the neuroactive steroid concentration is at least about 5 mg/mL. In some embodiments, the neuroactive steroid concentration is at most about 800 mg/mL. In some embodiments, the neuroactive steroid concentration is about 5 mg/mL to about 10 mg/mL, about 5 mg/mL to about 50 mg/mL, about 5 mg/mL to about 100 mg/mL, about 5 mg/mL to about 200 mg/mL, about 5 mg/mL to about 400 mg/mL, about 5 mg/mL to about 800 mg/mL, about 10 mg/mL to about 50 mg/mL, about 10 mg/mL to about 100 mg/mL, about 10 mg/mL to about 200 mg/mL, about 10 mg/mL to about 400 mg/mL, about 10 mg/mL to about 800 mg/mL, about 50 mg/mL to about 100 mg/mL, about 50 mg/mL to about 200 mg/mL, about 50 mg/mL to about 400 mg/mL, about 50 mg/mL to about 800 mg/mL, about 100 mg/mL to about 200 mg/mL, about 100 mg/mL to about 400 mg/mL, about 100 mg/mL to about 800 mg/mL, about 200 mg/mL to about 400 mg/mL, about 200 mg/mL to about 800 mg/mL, or about 400 mg/mL to about 800 mg/mL. In some embodiments, the neuroactive steroid concentration is about 5 mg/mL, about 10 mg/mL, about 50 mg/mL, about 100 mg/mL, about 200 mg/mL, about 400 mg/mL, or about 800 mg/mL. In some embodiments, the neuroactive steroid concentration is about 300 mg/mL.

In some embodiments, the neuroactive steroid maintains a plasma concentration of more than about 10%, 15%, 20%, 25%, or 30% of the Cₘₐₓ for at least about 10, 20, 30, 40, 50, or 60 days. The neuroactive steroid maintains a plasma concentration of more than about 10% the Cₘₐₓ for at least about 10 days in one embodiment, 15% the Cₘₐₓ for at least about 10 days in another embodiment, 20% the Cₘₐₓ for at least about 10 days in yet another embodiment, 25% the Cₘₐₓ for at least about 10 days in yet another embodiment, 35% the Cₘₐₓ for at least about 10 days in yet another embodiment, 10% the Cₘₐₓ for at least about 20 days in another embodiment, 15% the Cₘₐₓ for at least about 20 days in another embodiment, 25% the Cₘₐₓ for at least about 20 days in yet another embodiment, 35% the Cₘₐₓ for at least about 20 days in yet another embodiment, 10% the Cₘₐₓ for at least about 30 days in another embodiment, 15% the Cₘₐₓ for at least about 30 days in another embodiment, 25% the Cₘₐₓ for at least about 30 days in yet another embodiment, 35% the Cₘₐₓ for at least about 30 days in yet another embodiment, 10% the Cₘₐₓ for at least about 40 days in another embodiment, 15% the Cₘₐₓ for at least about 40 days in another embodiment, 25% the Cₘₐₓ for at least about 40 days in yet another embodiment, 35% the Cₘₐₓ for at least about 40 days in yet another embodiment, 10% the Cₘₐₓ for at least about 50 days in another embodiment, 15% the Cₘₐₓ for at least about 50 days in another embodiment, 25% the Cₘₐₓ for at least about 50 days in yet another embodiment, 35% the Cₘₐₓ for at least about 50 days in yet another embodiment, 10% the Cₘₐₓ for at least about 60 days in another embodiment, 15% the Cₘₐₓ for at least about 60 days in another embodiment, 25% the Cₘₐₓ for at least about 60 days in yet another embodiment, 35% the Cₘₐₓ for at least about 60 days in yet another embodiment, 10% the Cₘₐₓ for at least about 60 or more days in another embodiment, 15% the Cₘₐₓ for at least about 60 or more days in another embodiment, 25% the Cₘₐₓ for at least about 60 or more days in yet another embodiment or 35% the Cₘₐₓ for at least about 60 or more days in yet another embodiment. In one particular embodiment, the neuroactive steroid maintains a plasma concentration of more than about 15% of the Cₘₐₓ for at least about 30 days.

In some embodiments, the Cₘₐₓ is about 1 ng/mL to about 100 ng/mL. In some embodiments, the Cₘₐₓ is at least about 1 ng/mL. In some embodiments, the Cₘₐₓ is at most about 100 ng/mL. In some embodiments, the Cₘₐₓ is about 1 ng/mL to about 10 ng/mL, about 1 ng/mL to about 20 ng/mL, about 1 ng/mL to about 40 ng/mL, about 1 ng/mL to about 60 ng/mL, about 1 ng/mL to about 80 ng/mL, about 1 ng/mL to about 100 ng/mL, about 10 ng/mL to about 20 ng/mL, about 10 ng/mL to about 40 ng/mL, about 10 ng/mL to about 60 ng/mL, about 10 ng/mL to about 80 ng/mL, about 10 ng/mL to about 100 ng/mL, about 20 ng/mL to about 40 ng/mL, about 20 ng/mL to about 60 ng/mL, about 20 ng/mL to about 80 ng/mL, about 20 ng/mL to about 100 ng/mL, about 40 ng/mL to about 60 ng/mL, about 40 ng/mL to about 80 ng/mL, about 40 ng/mL to about 100 ng/mL, about 60 ng/mL to about 80 ng/mL, about 60 ng/mL to about 100 ng/mL, or about 80 ng/mL to about 100 ng/mL. In some embodiments, the Cₘₐₓ is about 1 ng/mL, about 10 ng/mL, about 20 ng/mL, about 40 ng/mL, about 60 ng/mL, about 80 ng/mL, or about 100 ng/mL. In particular examples, the Cₘₐₓ is in a range of from 20 to 90 ng/mL.

In some embodiments, the single dose is in a range of from 3 to about 5 mg per kilogram of body weight, and/or the neuroactive steroid can maintain a plasma concentration of more than about 10 ng/mL for at least about 5 days. In particular embodiments, the neuroactive steroid can maintain a plasma concentration of more than 10, 20, 25, 30, 40, 50, 60, 70, 80, 90, 100 ng/mL for at least about 10, 20, 30, 40, 50, or 60 days. In some embodiments, the neuroactive steroid can maintain a plasma concentration of more than 10 ng/mL for at least about 10 days in one embodiment, more than 20 ng/mL for at least about 10 days in another embodiment, more than 30 ng/mL for at least about 10 days in yet another embodiment, more than 40 ng/mL for at least about 10 days in yet another embodiment, more than 50 ng/mL for at least about 10 days in yet another embodiment, more than 60 ng/mL for at least about 10 days in yet another embodiment, more than 70 ng/mL for at least about 10 days in yet another embodiment, more than 80 ng/mL for at least about 10 days in yet another embodiment, more than 90 ng/mL for at least about 10 days in yet another embodiment, more than 100 ng/mL for at least about 10 days in yet another embodiment, more than 10 ng/mL for at least about 20 days in one embodiment, more than 20 ng/mL for at least about 20 days in another embodiment, more than 30 ng/mL for at least about 20 days in yet another embodiment, more than 40 ng/mL for at least about 20 days in yet another embodiment, more than 50 ng/mL for at least about 20 days in yet another embodiment, more than 60 ng/mL for at least about 20 days in yet another embodiment, more than 70 ng/mL for at least about 20 days in yet another embodiment, more than 80 ng/mL for at least about 20 days in yet another embodiment, more than 90 ng/mL for at least about 20 days in yet another embodiment, more than 100 ng/mL for at least about 20 days in yet another embodiment, more than 10 ng/mL for at least about 30 days in one embodiment, more than 20 ng/mL for at least about 30 days in another embodiment, more than 30 ng/mL for at least about 30 days in yet another embodiment, more than 40 ng/mL for at least about 30 days in yet another embodiment, more than 50 ng/mL for at least about 30 days in yet another embodiment, more than 60 ng/mL for at least about 30 days in yet another embodiment, more than 70 ng/mL for at least about 30 days in yet another embodiment, more than 80 ng/mL for at least about 30 days in yet another embodiment, more than 90 ng/mL for at least about 30 days in yet another embodiment, more than 100 ng/mL for at least about 30 days in yet another embodiment, more than 10 ng/mL for at least about 40 days in one embodiment, more than 20 ng/mL for at least about 40 days in another embodiment, more than 30 ng/mL for at least about 40 days in yet another embodiment, more than 40 ng/mL for at least about 40 days in yet another embodiment, more than 50 ng/mL for at least about 40 days in yet another embodiment, more than 60 ng/mL for at least about 40 days in yet another embodiment, more than 70 ng/mL for at least about 40 days in yet another embodiment, more than 80 ng/mL for at least about 40 days in yet another embodiment, more than 90 ng/mL for at least about 40 days in yet another embodiment, more than 100 ng/mL for at least about 40 days in yet another embodiment, more than 10 ng/mL for at least about 50 days in one embodiment, more than 20 ng/mL for at least about 50 days in another embodiment, more than 30 ng/mL for at least about 50 days in yet another embodiment, more than 40 ng/mL for at least about 50 days in yet another embodiment, more than 50 ng/mL for at least about 50 days in yet another embodiment, more than 60 ng/mL for at least about 50 days in yet another embodiment, more than 70 ng/mL for at least about 50 days in yet another embodiment, more than 80 ng/mL for at least about 50 days in yet another embodiment, more than 90 ng/mL for at least about 50 days in yet another embodiment, more than 100 ng/mL for at least about 50 days in yet another embodiment, more than 10 ng/mL for at least about 60 days in one embodiment, more than 20 ng/mL for at least about 60 days in another embodiment, more than 30 ng/mL for at least about 60 days in yet another embodiment, more than 40 ng/mL for at least about 60 days in yet another embodiment, more than 50 ng/mL for at least about 60 days in yet another embodiment, more than 60 ng/mL for at least about 60 days in yet another embodiment, more than 70 ng/mL for at least about 60 days in yet another embodiment, more than 80 ng/mL for at least about 60 days in yet another embodiment, more than 90 ng/mL for at least about 60 days in yet another embodiment, more than 100 ng/mL for at least about 60 days in yet another embodiment, more than 10 ng/mL for at least about 60 or more days in one embodiment, more than 20 ng/mL for at least about 60 or more days in another embodiment, more than 30 ng/mL for at least about 60 or more days in yet another embodiment, more than 40 ng/mL for at least about 60 or more days in yet another embodiment, more than 50 ng/mL for at least about 60 or more days in yet another embodiment, more than 60 ng/mL for at least about 60 or more days in yet another embodiment, more than 70 ng/mL for at least about 60 or more days in yet another embodiment, more than 80 ng/mL for at least about 60 or more days in yet another embodiment, more than 90 ng/mL for at least about 60 or more days in yet another embodiment and more than 100 ng/mL for at least about 60 or more days in yet another embodiment. In one further embodiment, the neuroactive steroid can maintain a plasma concentration of more than 20 ng/mL for at least about 30 days.

In some embodiments, the pharmaceutical composition releases less than about 5%-50% of the neuroactive steroid within about 1 hour of the single dose of the pharmaceutical composition by intramuscular or subcutaneous injection. Particularly, the pharmaceutical composition can release less than about 5%-50% in one embodiment, 10%-50% in another embodiment, 15%-50% in yet another embodiment, 20%-50% in yet another embodiment, 25%-50% in yet another embodiment, 30%-50% in yet another embodiment, 40%-50% in yet another embodiment and 45%-50% in yet another embodiment, of the neuroactive steroid into plasma of a subject within about 1 hour of the single dose of the pharmaceutical composition administered to the subject by intramuscular or subcutaneous injection. The percentage of release is based on measured plasma concentration of the neuroactive steroid and the total amount of the neuroactive steroid in the single dose of the pharmaceutical composition administered to the subject.

In some embodiments, the pharmaceutical composition can have a relative bioavailability (Bioavailability_{IM/SC}/Bioavailability_{IV}) of about 2%-50% at 24 hours after the single dose by intramuscular or subcutaneous injection, in comparison to the same dose by intravenous administration. In some embodiments, the relative bioavailability is about 2% to about 50%. In some embodiments, the relative bioavailability is at least about 2%. In some embodiments, the relative bioavailability is at most about 50%. In some embodiments, the relative bioavailability is about 2% to about 5%, about 2% to about 10%, about 2% to about 20%, about 2% to about 30%, about 2% to about 40%, about 2% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50%. In some embodiments, the relative bioavailability is about 2%, about 5%, about 10%, about 20%, about 30%, about 40%, or about 50%.

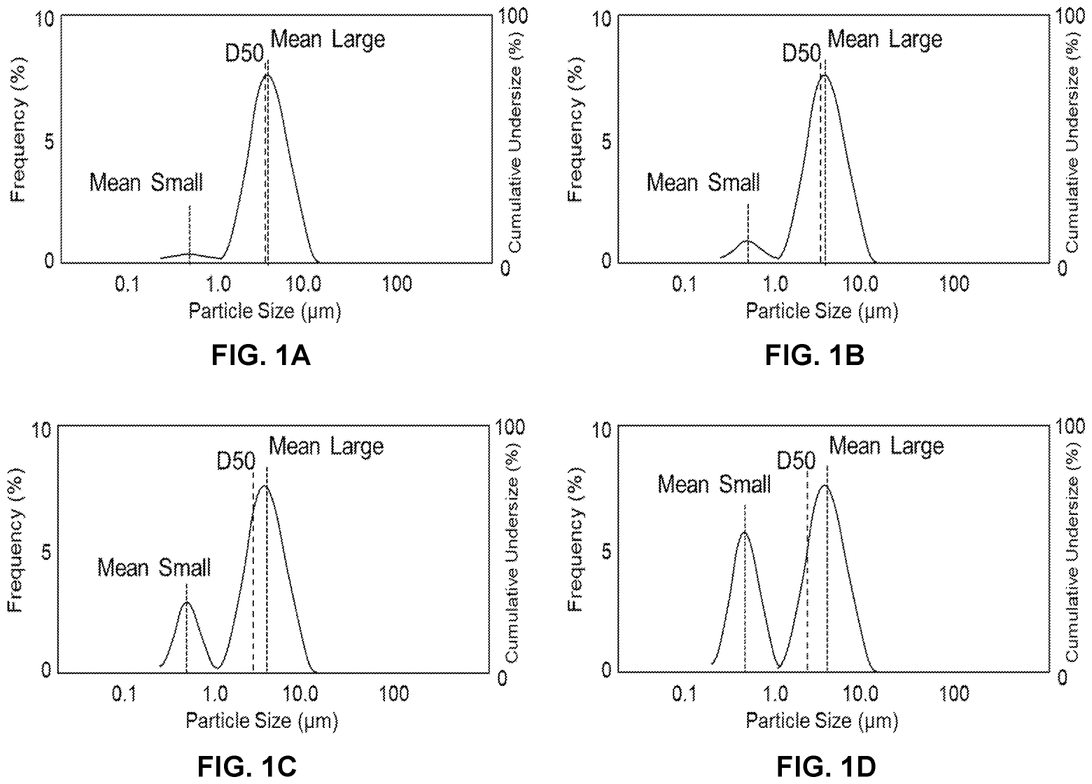

In some embodiments, the disclosed pharmaceutical composition comprises particles comprising at least one neuroactive steroid and one or more pharmaceutical acceptable excipients, wherein the neuroactive steroid is a positive modulator of γaminobutyric acid type A (GABA_{A}) receptors; wherein, particles comprise large particles having a particle size in a range of from about 1.5 μm to about 15 μm and small particles having a particle size in a range of from about 0.2 μm to about 1.5 μm; and wherein, about 0.01% to about 50% of the particles are small particles and about 50% to 99.99% of the particles are large particles, percentage based on the total weight of the particles. In any of embodiments or examples of the pharmaceutical composition, the particles can be stabilized particles disclosed herein. Schematic illustrations of exemplary particle size distributions are shown in FIG. 1A-FIG. 1D. FIG. 1A: a pharmaceutical composition comprises minimum amounts, such as less than 1% of small particles and mostly large particles. FIG. 1B: A pharmaceutical composition comprises some small particles and mostly large particles. FIG. 1C: A pharmaceutical composition comprises increasing amounts of small particles and large particles. FIG. 1D: A pharmaceutical composition comprises comparable amounts of small particles and large particles. D50=mass-median-diameter (MMD), wherein 50% of particles are below and 50% of particles are above a given diameter. Mean Large=mean particle size of the large particles. Mean Small=mean particle size of the small particles. In a further example, a mean particle size can be about 3 μm.

In some embodiments, the disclosed pharmaceutical composition comprises a pharmaceutically effective amount of a neuroactive steroid, wherein the neuroactive steroid is a positive modulator of gamma-aminobutyric acid type A (GABA_{A}) receptor; wherein the neuroactive steroid reaches a maximum plasma concentration (Cₘₐₓ) in a subject in about 30 minutes to 6 hours and maintains a plasma concentration of the neuroactive steroid more than about 5% of the Cₘₐₓ for at least about 5 days, after a single dose of the pharmaceutical composition is administered to the subject by intravenous (IV) injection, intramuscular (IM) injection, subcutaneous (SC) injection or a combination thereof; wherein, the pharmaceutical composition comprising particles of the neuroactive steroid; wherein, the particles comprise large particles having a particle size in a range of from about 1.5 μm to about 15 μm and small particles having a particle size in a range of from about 0.2 μm to about 1.5 μm; and wherein, about 0.01% to about 50% of the particles are small particles and about 50% to 99.99% of the particles are large particles, percentage based on the total weight of the particles.

In some embodiments, the maximum plasma concentration (Cₘₐₓ) is measured from specimens from the subject.

In some embodiments, the particles can have a D50 in a range of from 1.2 μm to about 6.0 μm. The D50 is 1.2 μm in one embodiment, 1.3 μm in another embodiment, 1.4 μm in yet another embodiment, 1.5 μm in yet another embodiment, 1.6 μm in yet another embodiment, 1.7 μm in yet another embodiment, 1.8 μm in yet another embodiment, 1.9 μm in yet another embodiment, 2.0 μm in yet another embodiment, 2.2 μm in yet another embodiment, 2.4 μm in yet another embodiment, 2.6 μm in yet another embodiment, 2.8 μm in yet another embodiment, 3.0 μm in yet another embodiment, 3.5 μm in yet another embodiment, 4.0 μm in yet another embodiment, 4.5 μm in yet another embodiment, 5.0 μm in yet another embodiment, 5.5 μm in yet another embodiment, 6.0 μm in yet another embodiment, or any one value in the range of from 1.2 μm through 6.0 μm in a further embodiment.

The neuroactive steroid of the disclosed pharmaceutical composition comprises tetrahydrodeoxycorticosterone (THDOC), androstane, androstane 3α-androstanediol, cholestane cholesterol, pregnane, pregnane pregnanolone (eltanolone), allopregnanolone, brexanolone, ganaxolone, zuranolone (SAGE-217) or a combination thereof. For example, in some embodiments, the neuroactive steroid is brexanolone.

In some embodiments, the large particles have a mean particle size in a range of from 2.0 to 6.0 μm in one embodiment, a mean particle size in a range of from 2.0 to 4.0 μm in another embodiment, a mean particle size of about 3 μm in yet another embodiment, a mean particle size in a range of from 3.0 to 5.0 μm in another embodiment, a mean particle size in a range of from 0.4 to 1.3 μm in yet another embodiment and a mean particle size in a range of from 0.5 to 0.9 μm in a further embodiment.

In some embodiments, the subject is a lactating woman who is breastfeeding and the disease is postpartum depression (PPD). The lactating woman can be in need for treatment or prevention of PPD. In some embodiments, a lactating woman is administered the NAS disclosed herein for treatment or prevention of PPD and co-administered the steroid composition for treatment or prevention of ISR. Any of the NAS disclosed above and hereafter can be suitable. The steroid composition comprises corticosteroid disclosed above and hereafter. In some embodiments, the corticosteroid is selected from the group consisting of methylprednisolone, methylprednisolone acetate, cortisone, cortisone acetate, hydrocortisone, hydrocortisone aceponate, hydrocortisone acetate, hydrocortisone buteprate, hydrocortisone butyrate, hydrocortisone valerate, prednisolone, prednisone, and a combination thereof. In some embodiments, the corticosteroid is methylprednisolone or methylprednisolone acetate. Cortisone is a normal component of breastmilk that passes from the mother's bloodstream into milk and its effect in breastmilk after exogenous administration in pharmacologic amounts has not been fully studied (Drugs and Lactation Database (LactMed) [Internet]. Bethesda (Md.): National Library of Medicine (US); 2006-. Cortisone. [Updated 2018 Oct. 31]). Some studies also show that cortisone injection is safe and efficient for pain relief in lactating women (Avci, S., et al., J Hand Surg; 27A:322-324, 2002). In some embodiments, the steroid composition is admixed with the NAS prior to injection and administered together at an injection site. In some embodiments, the steroid composition is co-formulated with the NAS and administered together at the injection site. In some embodiments, the steroid composition is co-formulated with the NAS to form a therapeutic composition as disclosed herein. The steroid composition and the NAS can be co-formulated with one or more pharmaceutical acceptable excipients. Excipients disclosed herein is suitable. The steroid composition co-formulated with the NAS is administered to a subject in need thereof via one or more injections at one or more injection sites. In some embodiments, the steroid composition is co-administered to the subject within 0 to 60 minutes before or after the NAS is administered to the subject.

In some embodiments, the subject is in lactation that can include, for example, a woman who is lactating, nursing or breastfeeding. The subject is a woman in lactation after giving birth. The subject can also be a woman in induced lactation. Based on CDC guideline (https://www.cdc.gov/breastfeeding/breastfeeding-special-circumstances/maternal-or-infant-illnesses/postpartum-depression.html) and a recent study, allopregnanolone (ALLO) concentrations in milk and plasma are low and that the brexanolone is associated with low risk to breastfed infants (Hoffmann, et al., Obstetrics & Gynecology, Volume 133, p 115S, May 2019, and Hoffmann, et al., American Journal of Obstetrics & Gynecology, 5554 Supplement to JANUARY 2019).

In further cases, the process can further comprise administering to the subject a therapeutically effective amount of one or more therapeutic agents. The therapeutic agents comprises one or more pain management medications, anti-inflammation medications, or a combination thereof. In one example, the therapeutic agents comprises pain management medication. In another example, the therapeutic agents comprises anti-inflammation medications, such as anti-histamine drugs, such as diphenhydramine (Benadryl), promethazine (Phenergan), hydroxyzine hydrochloride (Restall, Vistacot), or dimenhydrinate (Dramamine, Driminate). In some embodiments, the therapeutic agents are administered to the subject orally or via injection.

The present disclosure is also directed to a method for treating a central nervous system (CNS) disorder related to gamma-aminobutyric acid type A (GABA_{A}) receptor modulation in a subject in need thereof. The method can comprise administering a neuroactive steroid (NAS) that is a positive modulator of GABA_{A} receptor, or a pharmaceutically acceptable salt thereof, and a steroid composition to the subject via injection, wherein the neuroactive steroid (NAS) and the steroid composition is administered to the subject via intramuscular (IM) injection or subcutaneous injection (SC), e.g., at at least one injection site.

In some embodiments, the steroid composition is 1) co-formulated with the neuroactive steroid (NAS) and administered together to the subject via injection, 2) the steroid composition is admixed with the neuroactive steroid (NAS) and administered together to the subject via injection, 3) the steroid composition is co-administered with the neuroactive steroid (NAS), or 4) a combination thereof. In some embodiments, the steroid composition is admixed with the neuroactive steroid (NAS) prior to injection and administered together at the injection site. In some embodiments, the steroid composition is co-formulated with the neuroactive steroid (NAS) and administered together at the injection site. In some embodiments, the steroid composition is co-administered to the subject within 0 to 60 minutes before or after the neuroactive steroid (NAS) is administered to the subject.

In some embodiments, the steroid composition is co-administered to the subject at a co-administration site that is within 0 to 10 centimeters from the injection site. In some embodiments, the steroid composition is administered to the subject at the co-administration site that is within 0 to 5 centimeters, 0 to 4 centimeters, 0 to 3 centimeters, 0 to 2 centimeters, or 0 to 1 centimeter, from the injection site.

In some embodiments, the CNS disorder comprises depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, or a combination thereof.

In some embodiments, the CNS disorder is selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

In some embodiments, a subject treated for a depressive disorder with the presently disclosed methods experiences a reduction of depression as measured by a decrease in their HAM-D score. HAM-D is a depression rating scale consisting of 17 items, with the total score of the 17 items ranging from 0 to 54, with higher scores indicating greater depression. In some embodiments, after treatment the subject experiences a reduction of depression characterized by a 1- to 10-point (e.g., a 1-point, 2-point, 3-point, 4-point, 5-point, 6-point, 7-point, 8-point, 9-point, or 10-point) decrease in a HAM-D score compared to their baseline score prior to treatment. In some embodiments, the HAM-D score is determined at day 3, 8, 15, 30, and 45 after treatment.

In some embodiments, a subject treated for a depressive disorder with the presently disclosed methods experiences a reduction of depression as measured by a decrease in their Montgomery Asberg Depression Rating Scale (MADRS). MADRS is a depression rating scale consisting of 10 items, each rated 0 to 6. The total score of the 10 items ranges from 0 to 60, with higher scores indicating greater depression. In some embodiments, after treatment the subject experiences a reduction of depression characterized by a 1- to 10-point (e.g., a 1-point, 2-point, 3-point, 4-point, 5-point, 6-point, 7-point, 8-point, 9-point, or 10-point) decrease in a MADRS score compared to their baseline score prior to treatment. In some embodiments, the MADRS score is determined at day 3, 8, 15, 30, and 45 after treatment.

In some embodiments, the neuroactive steroid (NAS) comprises brexanolone, long-acting brexanolone, slow-releasing brexanolone, ganaxolone, zuranolone, a prodrug thereof, or a combination thereof.

In some embodiments, the neuroactive steroid (NAS) is selected from the group consisting of brexanolone, long-acting brexanolone, slow-releasing brexanolone, ganaxolone, zuranolone, a prodrug thereof, and a combination thereof.

In some embodiments, the neuroactive steroid (NAS) is selected from the group consisting of brexanolone, long-acting brexanolone, slow-releasing brexanolone, a prodrug thereof, and a combination thereof.

In some embodiments, the neuroactive steroid (NAS) comprises a brexanolone crystalline form, e.g., brexanolone polymorph Form A.

In some embodiments, the NAS comprises brexanolone aqueous suspension BRII-296, which is a long-acting brexanolone.

In some embodiments, the neuroactive steroid composition is administered to the subject in a single dose comprising about 0.01 to 50 mg NAS per kilogram of body weight of the subject. In some embodiments, the neuroactive steroid composition is administered to the subject in a single dose comprising from 0.01 to 1 mg, 0.01 to 2 mg, 0.01 to 4 mg, 0.01 to 5 mg, 0.01 to 8 mg, 0.01 to 10 mg, 0.01 to 20 mg, 0.01 to 30 mg, 0.01 to 40 mg and 0.01 to 50 mg of the neuroactive steroid per kilogram of body weight of the subject. In some embodiments, the single dose comprises from about 50 mg to 1,000 mg of the neuroactive steroid. In further examples, the single dose comprises from about 50 mg to about 600 mg of the neuroactive steroid. The single NAS dose can be packaged in an NAS packing unit. One or more NAS packing units can be used for an injection.

All ranges and sub-ranges disclosed above and hereafter in this disclosure can be suitable.

In some embodiments, the steroid composition is administered to a subject in a single site dose comprising from 10 to 100 mg of the steroid composition for each injection site and the pharmaceutical composition is administered to a subject in single site dose comprising of from 30 mg to 1200 mg of the neuroactive steroid for each injection site. The single site dose of the steroid composition is from 10 to 100 mg in one embodiment, 20 to 100 mg in another embodiment, 30 to 100 mg in yet another embodiment, 40 to 100 mg in yet another embodiment, 50 to 100 mg in yet another embodiment, 60 to 100 mg in yet another embodiment, 70 to 100 mg in yet another embodiment, 80 to 100 mg in yet another embodiment, and 90 to 100 mg in yet another embodiment, per injection site. In some embodiments, the single site dose of the steroid composition comprises from 30 to 40 mg, 40 mg, 40 to 50 mg, 50 mg, 50 to 60 mg, 60 mg, 60 to 70 mg, 70 mg, 70 to 80 mg, 80 mg, 80 to 90 mg, 90 mg, and 90 to 100 mg of the steroid (e.g., corticosteroid) per injection site. In some embodiments, the single site dose comprises from 30 mg to 1200 mg in one embodiment, 40 mg to 1200 mg in another embodiment, 50 to 1200 mg in yet another embodiment, 60 to 1200 mg in yet another embodiment, 70 to 1200 mg in yet another embodiment, 80 to 1200 in yet another embodiment, 90 to 1200 mg in yet another embodiment, 100 to 1200 in yet another embodiment, 200 to 1200 mg in yet another embodiment, 300 to 1200 mg in yet another embodiment, 400 to 1200 mg in yet another embodiment, 500 to 1200 mg in yet another embodiment, 600 to 1200 in yet another embodiment, 700 to 1200 mg in yet another embodiment, 800 to 1200 mg in yet another embodiment, 900 to 1200 mg in yet another embodiment, 1000 to 1200 mg in yet another embodiment, and 1100 to 1200 mg in yet another embodiment, of the neuroactive steroid, per injection site. In some embodiments, the single site dose of the pharmaceutical composition comprises from 100 to 800 mg, 200 to 800 mg, 300 to 800 mg, 400 to 800 mg, 500 to 800 mg, and 600 to 800 mg of the neuroactive steroid, per injection site. All ranges cited herein are inclusive. A combination of any of the aforementioned single site steroid dosage and the single site NAS dosage can be suitable. In some embodiments, a pre-mixed therapeutic composition comprises 10 to 100 mg of the steroid composition and 50 to 1200 mg of the neuroactive steroid. In some embodiments, a pre-mixed therapeutic composition comprises 20 to 40 mg of the steroid composition and 200 to 800 mg of the neuroactive steroid. The single site steroid dose can be administered using one or more dose units and the single site NAS dose can be administered using one or more dose units of the neuroactive steroid disclosed herein. In some embodiments, a 40 mg single site dose of the steroid (e.g., corticosteroid) and 600 mg single site NAS dose can be administered using 2 injections each comprising 20 mg of the steroid and 300 mg of the neuroactive steroid. The steroidal amounts described herein in the unit “mg” refer to an effective dose of the steroid, such as corticosteroid disclosed above and hereafter and/or any other active pharmaceutical ingredient (API), such as neuroactive steroid (NAS). As used herein throughout this disclosure, an amount of the steroid composition, such as a certain “mg of the steroid composition”, refers to the amount of the effective dose, i.e., mg, of the steroid, such as a corticosteroid disclosed herein. As used herein throughout this disclosure, an amount of the pharmaceutical composition, such as a certain “mg of the pharmaceutical composition”, refers to the amount of the effective dose, i.e., mg, of the active pharmaceutical ingredient (API), such as the neuroactive steroid (NAS) disclosed herein. It is understood that the amount of excipient(s) is not included in any of the terms, such as dose, dose, effective dose, unit, amount, weight, mg, ng, mg/mL, ng/mL, μg/mL, or other similar terms used herein. For any of the NAS prodrugs, the amount of each NAS prodrug dose can be converted to a respective single site NAS dose equivalent based on molecular weights and biological efficacy.

In some embodiments, the steroid composition is administered to a subject in a single site dose comprising from 10 to 100 mg of the steroid for each injection site and the pharmaceutical composition is administered to a subject in a single site dose comprising from 30 mg to 1200 mg of the neuroactive steroid (NAS) for each injection site.

In some embodiments, the steroid composition and the neuroactive steroid are formulated in an injection volume in a range of from 0.5 mL to 6 mL for each of the injection site. In some embodiments, the steroid composition and the neuroactive steroid are formulated in an injection volume in a range of from 0.5 mL to 1 mL in one embodiment, 0.5 mL to 2 mL in another embodiment, 0.5 mL to 3 mL in yet another embodiment, 0.5 mL to 4 mL in yet another embodiment, 0.5 mL to 5 mL in yet another embodiment, 0.5 mL to 6 mL in yet embodiment, 1 mL to 2 mL in yet another embodiment, 1 mL to 3 mL in yet another embodiment, 1 mL to 4 mL in yet another embodiment, 1 mL to 5 mL in yet another embodiment, 1 mL to 6 mL in yet another embodiment, 2 mL to 3 mL in yet another embodiment, 2 mL to 4 mL in yet another embodiment, 2 mL to 5 mL in yet another embodiment, 2 mL to 6 mL in yet another embodiment, 3 mL to 4 mL in yet another embodiment, 3 mL to 5 mL in yet another embodiment, and 3 mL to 6 mL in yet another embodiment, for each injection site. The steroid composition and the neuroactive steroid in the injection volume can be administered using one or more units or sub-volumes. In some embodiments, a 6 mL injection volume is administered using 2 injections each has a volume of 3 mL.

In some embodiments, the neuroactive steroid reaches a maximum plasma concentration (Cₘₐₓ) in about 30 minutes to 6 hours and maintains a plasma concentration of more than about 5% of the Cₘₐₓ for at least about 5 days, in the subject, after a single dose of the neuroactive steroid.

In some embodiments, the neuroactive steroid has a concentration of about 5 mg/mL to about 800 mg/mL. In some embodiments, the neuroactive steroid concentration is at least about 5 mg/mL. In some embodiments, the neuroactive steroid concentration is at most about 800 mg/mL. In some embodiments, the neuroactive steroid concentration is about 5 mg/mL to about 10 mg/mL, about 5 mg/mL to about 50 mg/mL, about 5 mg/mL to about 100 mg/mL, about 5 mg/mL to about 200 mg/mL, about 5 mg/mL to about 400 mg/mL, about 5 mg/mL to about 800 mg/mL, about 10 mg/mL to about 50 mg/mL, about 10 mg/mL to about 100 mg/mL, about 10 mg/mL to about 200 mg/mL, about 10 mg/mL to about 400 mg/mL, about 10 mg/mL to about 800 mg/mL, about 50 mg/mL to about 100 mg/mL, about 50 mg/mL to about 200 mg/mL, about 50 mg/mL to about 400 mg/mL, about 50 mg/mL to about 800 mg/mL, about 100 mg/mL to about 200 mg/mL, about 100 mg/mL to about 400 mg/mL, about 100 mg/mL to about 800 mg/mL, about 200 mg/mL to about 400 mg/mL, about 200 mg/mL to about 800 mg/mL, or about 400 mg/mL to about 800 mg/mL. In some embodiments, the neuroactive steroid concentration is about 5 mg/mL, about 10 mg/mL, about 50 mg/mL, about 100 mg/mL, about 200 mg/mL, about 400 mg/mL, or about 800 mg/mL.

In some embodiments, the neuroactive steroid is formulated to maintain a plasma concentration of more than about 10%, 15%, 20%, 25%, or 30% of the Cₘₐₓ for at least about 10, 20, 30, 40, 50, or 60 days. The neuroactive steroid is formulated to maintain a plasma concentration of more than about 10% the Cₘₐₓ for at least about 10 days in one embodiment, 15% the Cₘₐₓ for at least about 10 days in another embodiment, 20% the Cₘₐₓ for at least about 10 days in yet another embodiment, 25% the Cₘₐₓ for at least about 10 days in yet another embodiment, 35% the Cₘₐₓ for at least about 10 days in yet another embodiment, 10% the Cₘₐₓ for at least about 20 days in another embodiment, 15% the Cₘₐₓ for at least about 20 days in another embodiment, 25% the Cₘₐₓ for at least about 20 days in yet another embodiment, 35% the Cₘₐₓ for at least about 20 days in yet another embodiment, 10% the Cₘₐₓ for at least about 30 days in another embodiment, 15% the Cₘₐₓ for at least about 30 days in another embodiment, 25% the c for at least about 30 days in yet another embodiment, 35% the Cₘₐₓ for at least about 30 days in yet another embodiment, 10% the Cₘₐₓ for at least about 40 days in another embodiment, 15% the Cₘₐₓ for at least about 40 days in another embodiment, 25% the Cₘₐₓ for at least about 40 days in yet another embodiment, 35% the Cₘₐₓ for at least about 40 days in yet another embodiment, 10% the Cₘₐₓ for at least about 50 days in another embodiment, 15% the Cₘₐₓ for at least about 50 days in another embodiment, 25% the Cₘₐₓ for at least about 50 days in yet another embodiment, 35% the Cₘₐₓ for at least about 50 days in yet another embodiment, 10% the Cₘₐₓ for at least about 60 days in another embodiment, 15% the Cₘₐₓ for at least about 60 days in another embodiment, 25% the Cₘₐₓ for at least about 60 days in yet another embodiment, 35% the Cₘₐₓ for at least about 60 days in yet another embodiment, 10% the Cₘₐₓ for at least about 60 or more days in another embodiment, 15% the Cₘₐₓ for at least about 60 or more days in another embodiment, 25% the Cₘₐₓ for at least about 60 or more days in yet another embodiment or 35% the Cₘₐₓ for at least about 60 or more days in yet another embodiment. In one particular embodiment, the neuroactive steroid can maintain a plasma concentration of more than about 15% of the Cₘₐₓ for at least about 30 days.

In some embodiments, the Cₘₐₓ of the neuroactive steroid is about 1 ng/mL to about 100 ng/mL. In some embodiments, the Cₘₐₓ is at least about 1 ng/mL. In some embodiments, the Cₘₐₓ is at most about 100 ng/mL. In some embodiments, the Cₘₐₓ is about 1 ng/mL to about 10 ng/mL, about 1 ng/mL to about 20 ng/mL, about 1 ng/mL to about 40 ng/mL, about 1 ng/mL to about 60 ng/mL, about 1 ng/mL to about 80 ng/mL, about 1 ng/mL to about 100 ng/mL, about 10 ng/mL to about 20 ng/mL, about 10 ng/mL to about 40 ng/mL, about 10 ng/mL to about 60 ng/mL, about 10 ng/mL to about 80 ng/mL, about 10 ng/mL to about 100 ng/mL, about 20 ng/mL to about 40 ng/mL, about 20 ng/mL to about 60 ng/mL, about 20 ng/mL to about 80 ng/mL, about 20 ng/mL to about 100 ng/mL, about 40 ng/mL to about 60 ng/mL, about 40 ng/mL to about 80 ng/mL, about 40 ng/mL to about 100 ng/mL, about 60 ng/mL to about 80 ng/mL, about 60 ng/mL to about 100 ng/mL, or about 80 ng/mL to about 100 ng/mL. In some embodiments, the Cₘₐₓ is about 1 ng/mL, about 10 ng/mL, about 20 ng/mL, about 40 ng/mL, about 60 ng/mL, about 80 ng/mL, or about 100 ng/mL. In particular examples, the Cₘₐₓ is in a range of from 20 to 90 ng/mL.

In some embodiments, the single dose of the pharmaceutical compositions comprises from 3 to about 5 mg of NAS per kilogram of body weight, and/or the neuroactive steroid maintains a plasma concentration of more than about 10 ng/mL for at least about 5 days. In particular embodiments, the neuroactive steroid maintains a plasma concentration of more than 10, 20, 25, 30, 40, 50, 60, 70, 80, 90, 100 ng/mL for at least about 10, 20, 30, 40, 50, or 60 days. In some embodiments, the neuroactive steroid maintains a plasma concentration of more than 10 ng/mL for at least about 10 days in one embodiment, more than 20 ng/mL for at least about 10 days in another embodiment, more than 30 ng/mL for at least about 10 days in yet another embodiment, more than 40 ng/mL for at least about 10 days in yet another embodiment, more than 50 ng/mL for at least about 10 days in yet another embodiment, more than 60 ng/mL for at least about 10 days in yet another embodiment, more than 70 ng/mL for at least about 10 days in yet another embodiment, more than 80 ng/mL for at least about 10 days in yet another embodiment, more than 90 ng/mL for at least about 10 days in yet another embodiment, more than 100 ng/mL for at least about 10 days in yet another embodiment, more than 10 ng/mL for at least about 20 days in one embodiment, more than 20 ng/mL for at least about 20 days in another embodiment, more than 30 ng/mL for at least about 20 days in yet another embodiment, more than 40 ng/mL for at least about 20 days in yet another embodiment, more than 50 ng/mL for at least about 20 days in yet another embodiment, more than 60 ng/mL for at least about 20 days in yet another embodiment, more than 70 ng/mL for at least about 20 days in yet another embodiment, more than 80 ng/mL for at least about 20 days in yet another embodiment, more than 90 ng/mL for at least about 20 days in yet another embodiment, more than 100 ng/mL for at least about 20 days in yet another embodiment, more than 10 ng/mL for at least about 30 days in one embodiment, more than 20 ng/mL for at least about 30 days in another embodiment, more than 30 ng/mL for at least about 30 days in yet another embodiment, more than 40 ng/mL for at least about 30 days in yet another embodiment, more than 50 ng/mL for at least about 30 days in yet another embodiment, more than 60 ng/mL for at least about 30 days in yet another embodiment, more than 70 ng/mL for at least about 30 days in yet another embodiment, more than 80 ng/mL for at least about 30 days in yet another embodiment, more than 90 ng/mL for at least about 30 days in yet another embodiment, more than 100 ng/mL for at least about 30 days in yet another embodiment, more than 10 ng/mL for at least about 40 days in one embodiment, more than 20 ng/mL for at least about 40 days in another embodiment, more than 30 ng/mL for at least about 40 days in yet another embodiment, more than 40 ng/mL for at least about 40 days in yet another embodiment, more than 50 ng/mL for at least about 40 days in yet another embodiment, more than 60 ng/mL for at least about 40 days in yet another embodiment, more than 70 ng/mL for at least about 40 days in yet another embodiment, more than 80 ng/mL for at least about 40 days in yet another embodiment, more than 90 ng/mL for at least about 40 days in yet another embodiment, more than 100 ng/mL for at least about 40 days in yet another embodiment, more than 10 ng/mL for at least about 50 days in one embodiment, more than 20 ng/mL for at least about 50 days in another embodiment, more than 30 ng/mL for at least about 50 days in yet another embodiment, more than 40 ng/mL for at least about 50 days in yet another embodiment, more than 50 ng/mL for at least about 50 days in yet another embodiment, more than 60 ng/mL for at least about 50 days in yet another embodiment, more than 70 ng/mL for at least about 50 days in yet another embodiment, more than 80 ng/mL for at least about 50 days in yet another embodiment, more than 90 ng/mL for at least about 50 days in yet another embodiment, more than 100 ng/mL for at least about 50 days in yet another embodiment, more than 10 ng/mL for at least about 60 days in one embodiment, more than 20 ng/mL for at least about 60 days in another embodiment, more than 30 ng/mL for at least about 60 days in yet another embodiment, more than 40 ng/mL for at least about 60 days in yet another embodiment, more than 50 ng/mL for at least about 60 days in yet another embodiment, more than 60 ng/mL for at least about 60 days in yet another embodiment, more than 70 ng/mL for at least about 60 days in yet another embodiment, more than 80 ng/mL for at least about 60 days in yet another embodiment, more than 90 ng/mL for at least about 60 days in yet another embodiment, more than 100 ng/mL for at least about 60 days in yet another embodiment, more than 10 ng/mL for at least about 60 or more days in one embodiment, more than 20 ng/mL for at least about 60 or more days in another embodiment, more than 30 ng/mL for at least about 60 or more days in yet another embodiment, more than 40 ng/mL for at least about 60 or more days in yet another embodiment, more than 50 ng/mL for at least about 60 or more days in yet another embodiment, more than 60 ng/mL for at least about 60 or more days in yet another embodiment, more than 70 ng/mL for at least about 60 or more days in yet another embodiment, more than 80 ng/mL for at least about 60 or more days in yet another embodiment, more than 90 ng/mL for at least about 60 or more days in yet another embodiment and more than 100 ng/mL for at least about 60 or more days in yet another embodiment. In one further embodiment, the neuroactive steroid maintains a plasma concentration of more than 20 ng/mL for at least about 30 days.

In some embodiments, the therapeutic composition releases less than about 5%-50% of the neuroactive steroid within about 1 hour of the single dose of the neuroactive steroid by intramuscular or subcutaneous injection. Particularly, the therapeutic composition can release less than about 5%-50% in one example, 10%-50% in another example, 15%-50% in yet another example, 20%-50% in yet another example, 25%-50% in yet another example, 30%-50% in yet another example, 40%-50% in yet another example and 45%-50% in yet another example, of the neuroactive steroid into plasma of a subject within about 1 hour of the single dose of the neuroactive steroid administered to the subject by intramuscular or subcutaneous injection. The percentage of release is based on measured plasma concentration of the neuroactive steroid and the total amount of the neuroactive steroid in the single dose of the neuroactive steroid administered to the subject.

In some embodiments, the neuroactive steroid has a relative bioavailability (Bioavailability_{IM/SC}/Bioavailability_{IV}) of about 2%-50% at 24 hours after the single dose by intramuscular or subcutaneous injection, in comparison to the same dose by intravenous administration. In some embodiments, the relative bioavailability is about 2% to about 50%. In some embodiments, the relative bioavailability is at least about 2%. In some embodiments, the relative bioavailability is at most about 50%. In some embodiments, the relative bioavailability is about 2% to about 5%, about 2% to about 10%, about 2% to about 20%, about 2% to about 30%, about 2% to about 40%, about 2% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50%. In some embodiments, the relative bioavailability is about 2%, about 5%, about 10%, about 20%, about 30%, about 40%, or about 50%.

In some embodiments, the disclosed therapeutic composition comprises particles comprising at least one neuroactive steroid and one or more pharmaceutical acceptable excipients, wherein the neuroactive steroid is a positive modulator of γaminobutyric acid type A (GABA_{A}) receptors; wherein, particles comprise large particles having a particle size in a range of from about 1.5 μm to about 15 μm and small particles having a particle size in a range of from about 0.2 μm to about 1.5 μm; and wherein, about 0.01% to about 50% of the particles are small particles and about 50% to 99.99% of the particles are large particles, percentage based on the total weight of the particles. In any of embodiments or examples of the neuroactive steroid, the particles can be stabilized particles disclosed herein.

In some embodiments, the disclosed therapeutic composition comprises a pharmaceutically effective amount of a neuroactive steroid, wherein the neuroactive steroid is a positive modulator of gamma-aminobutyric acid type A (GABA_{A}) receptor; wherein the neuroactive steroid reaches a maximum plasma concentration (Cₘₐₓ) in a subject in about 30 minutes to 6 hours and maintains a plasma concentration of the neuroactive steroid more than about 5% of the Cₘₐₓ for at least about 5 days, after a single dose of the neuroactive steroid is administered to the subject by intravenous (IV) injection, intramuscular (IM) injection, subcutaneous (SC) injection or a combination thereof; wherein, the therapeutic composition comprising particles of the neuroactive steroid; wherein, the particles comprise large particles having a particle size in a range of from about 1.5 μm to about 15 μm and small particles having a particle size in a range of from about 0.2 μm to about 1.5 μm; and wherein, about 0.01% to about 50% of the particles are small particles and about 50% to 99.99% of the particles are large particles, percentage based on the total weight of the particles.

In some embodiments, the maximum plasma concentration (Cₘₐₓ) is measured from specimens from the subject. In some embodiments, the specimen is blood of the subject.

In some embodiments, the particles have a D50 in a range of from 1.2 μm to about 6.0 μm. The D50 is 1.2 μm in one embodiment, 1.3 μm in another embodiment, 1.4 μm in yet another embodiment, 1.5 μm in yet another embodiment, 1.6 μm in yet another embodiment, 1.7 μm in yet another embodiment, 1.8 μm in yet another embodiment, 1.9 μm in yet another embodiment, 2.0 μm in yet another embodiment, 2.2 μm in yet another embodiment, 2.4 μm in yet another embodiment, 2.6 μm in yet another embodiment, 2.8 μm in yet another embodiment, 3.0 μm in yet another embodiment, 3.5 μm in yet another embodiment, 4.0 μm in yet another embodiment, 4.5 μm in yet another embodiment, 5.0 μm in yet another embodiment, 5.5 μm in yet another embodiment, 6.0 μm in yet another embodiment, or any one value in the range of from 1.2 μm through 6.0 μm in a further embodiment.

The neuroactive steroid of the disclosed therapeutic composition comprises tetrahydrodeoxycorticosterone (THDOC), androstane, androstane 3α-androstanediol, cholestane cholesterol, pregnane, pregnane pregnanolone (eltanolone), allopregnanolone, brexanolone, ganaxolone, zuranolone (SAGE-217) or a combination thereof. For example, the neuroactive steroid can be brexanolone.

In some embodiments, the large particles have a mean particle size in a range of from 2.0 to 6.0 μm in one embodiment, a mean particle size in a range of from 2.0 to 4.0 μm in another embodiment, a mean particle size of about 3 μm in yet another embodiment, a mean particle size in a range of from 3.0 to 5.0 μm in another embodiment, a mean particle size in a range of from 0.4 to 1.3 μm in yet another embodiment and a mean particle size in a range of from 0.5 to 0.9 μm in a further embodiment.

In some embodiments of the method, the steroid composition is administered to the subject within 0 to 60 minutes from the neuroactive steroid is administered to the subject. In some embodiments, the neuroactive steroid and the steroid composition are administered simultaneously.

In some embodiments, the neuroactive steroid and the steroid composition are mixed together, i.e., admixed, prior to administering to the subject at the injection site.

In some embodiments, the steroid composition is co-administered to the subject at a co-administration site that is within 0 to 10 centimeters from the injection site. In some embodiments, the steroid composition is administered to the subject at the co-administration site that is within 0 to 5 centimeters, 0 to 4 centimeters, 0 to 3 centimeters, 0 to 2 centimeters, or 0 to 1 centimeter, from the injection site.

In some embodiments, the steroid composition comprises a corticosteroid comprising alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate (DECADRON-LA), dexamethasone sodium (DECADRON, SOLUREX), fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate (DEPO-MEDROL), mometasone, mometasone furoate, prednicarbate, prednisolone, prednisone, tixocortol pivalate, triamcinolone acetonide, triamcinolone hexacetonide (ARISTOSPAN), triamcinolone acetonide (KENALOG), or a combination thereof.

In some embodiments, the corticosteroid comprises methylprednisolone acetate (DEPO-MEDROL), triamcinolone acetonide (KENALOG), triamcinolone hexacetonide (ARISTOSPAN), dexamethasone acetate (DECADRON-LA), dexamethasone sodium (DECADRON, SOLUREX), or a combination thereof. In some embodiments, the corticosteroid can be selected from the group consisting of methylprednisolone acetate (DEPO-MEDROL), triamcinolone acetonide (KENALOG), triamcinolone hexacetonide (ARISTOSPAN), dexamethasone acetate (DECADRON-LA), dexamethasone sodium (DECADRON, SOLUREX), and a combination thereof.

In some embodiments of the method, the steroid composition is administered to the subject in a single steroid dose in a range of from 0.01 to 50 mg of the steroid composition per kilogram of body weight of the subject. In some embodiments, the steroid composition is administered to the subject in a single steroid dose in a range of from 0.01 to 1 mg, 0.01 to 2 mg, 0.01 to 4 mg, 0.01 to 5 mg, 0.01 to 8 mg, 0.01 to 10 mg, 0.01 to 20 mg, 0.01 to 30 mg, 0.01 to 40 mg and 0.01 to 50 mg of the steroid composition per kilogram of body weight of the subject.

In some embodiments of the method, a single dose of the steroid composition comprises from about 10 mg to about 450 mg of the steroid. In certain embodiments, a single steroid dosage comprises one or more steroid packing units.

In some embodiments of the method, the neuroactive steroid and the steroid composition are mixed together prior to administering to the subject.

In some embodiments, the subject is a lactating woman who is breastfeeding and the disease is postpartum depression (PPD), and the corticosteroid is selected from any corticosteroids disclosed herein. In some embodiments, the corticosteroid is selected from the group consisting of methylprednisolone, methylprednisolone acetate, cortisone, cortisone acetate, hydrocortisone, hydrocortisone aceponate, hydrocortisone acetate, hydrocortisone buteprate, hydrocortisone butyrate, hydrocortisone valerate, prednisolone, prednisone, and a combination thereof.

### Therapeutic Compositions of the Disclosure

In some embodiments, the present disclosure is directed to therapeutic compositions for treating or preventing injection site reaction (ISR) in a subject in need thereof. In further embodiments, the present disclosure is directed to therapeutic compositions for treating a central nervous system (CNS) disorder in a subject in need thereof. In some embodiments, the present disclosure is directed to therapeutic compositions for treating a central nervous system (CNS) disorder and preventing injection site reaction (ISR) in a subject in need thereof.

In some embodiments, the therapeutic compositions disclosed herein comprise (i) a neuroactive steroid (NAS) that is a positive modulator of gamma-aminobutyric acid type A (GABA_{A}) receptor, or a pharmaceutically acceptable salt or composition thereof, (ii) a steroid composition, and (iii) one or more pharmaceutical acceptable excipients.

In some embodiments, the therapeutic composition is formulated for intravenous (IV) injection, intramuscular (IM) injection or subcutaneous injection (SC).

The therapeutic composition is formulated for treating the central nervous system (CNS) disorder and preventing the injection site reaction (ISR) in the subject.

In some embodiments, the pharmaceutical composition comprising a NAS is an aqueous composition. In some embodiments, the pharmaceutical composition is an aqueous suspension composition. In some embodiments, the pharmaceutical composition is the aqueous suspension composition provided in Table 1.

In some embodiments, the therapeutic composition is formulated to be administered to a subject in a single site dose comprising from 10 to 100 mg of the steroid for each of the injection site and a single site dose comprising from 30 mg to 1200 mg of the neuroactive steroid for each of the injection site. The single site dose of the steroid composition comprises from 10 to 100 mg of the steroid in one embodiment, 20 to 100 mg in another embodiment, 30 to 100 mg in yet another embodiment, 40 to 100 mg in yet another embodiment, 50 to 100 mg in yet another embodiment, 60 to 100 mg in yet another embodiment, 70 to 100 mg in yet another embodiment, 80 to 100 mg in yet another embodiment, and 90 to 100 mg in yet another embodiment, per injection site. In some embodiments, the single site dose of the steroid composition comprises from 30 to 40 mg, 40 mg, 40 to 50 mg, 50 mg, 50 to 60 mg, 60 mg, 60 to 70 mg, 70 mg, 70 to 80 mg, 80 mg, 80 to 90 mg, 90 mg, and 90 to 100 mg, of the steroid per injection site. In some embodiments, the single site dose of the pharmaceutical composition comprises from 30 mg to 1200 mg in one embodiment, 40 mg to 1200 mg in another embodiment, 50 to 1200 mg in yet another embodiment, 60 to 1200 mg in yet another embodiment, 70 to 1200 mg in yet another embodiment, 80 to 1200 in yet another embodiment, 90 to 1200 mg in yet another embodiment, 100 to 1200 in yet another embodiment, 200 to 1200 mg in yet another embodiment, 300 to 1200 mg in yet another embodiment, 400 to 1200 mg in yet another embodiment, 500 to 1200 mg in yet another embodiment, 600 to 1200 in yet another embodiment, 700 to 1200 mg in yet another embodiment, 800 to 1200 mg in yet another embodiment, 900 to 1200 mg in yet another embodiment, 1000 to 1200 mg in yet another embodiment, and 1100 to 1200 mg in yet another embodiment, of the neuroactive steroid, per injection site. In some embodiments, the single site dose of the pharmaceutical composition comprises from 100 to 800 mg, 200 to 800 mg, 300 to 800 mg, 400 to 800 mg, 500 to 800 mg, and 600 to 800 mg, of the neuroactive steroid, per injection site. All ranges cited herein are inclusive. A combination of any of the aforementioned single site steroid dose and the single site NAS dose is suitable. In some embodiments, a pre-mixed therapeutic composition comprises 10 to 100 mg of the steroid composition and 50 to 1200 mg of the neuroactive steroid. In some embodiments, a pre-mixed therapeutic composition comprises 20 to 40 mg of the steroid composition and 200 to 800 mg of the neuroactive steroid. The single site steroid dose can be administered using one or more dose units and the single site NAS dose can be administered using one or more dose units of the neuroactive steroid disclosed herein. In some embodiments, a 40 mg single site steroid dose of the steroid and 600 mg single site NAS dose is administered using 2 injections, each comprising 20 mg of the steroid and 300 mg of the neuroactive steroid. The weight unit “mg” used herein refers to the effective dose of the steroid, such as corticosteroid disclosed here and the active pharmaceutical ingredient (API), such as neuroactive steroid (NAS).

In some embodiments, the therapeutic composition is formulated in an injection volume ranging from 0.5 mL to 6 mL for each of injection site. In some embodiments, the therapeutic composition can be formulated in an injection volume ranging from 0.5 mL to 1 mL in one example, 0.5 mL to 2 mL in another example, 0.5 mL to 3 mL in yet another example, 0.5 mL to 4 mL in yet another example, 0.5 mL to 5 mL in yet another example, 0.5 mL to 6 mL in yet example, 1 mL to 2 mL in yet another example, 1 mL to 3 mL in yet another example, 1 mL to 4 mL in yet another example, 1 mL to 5 mL in yet another example, 1 mL to 6 mL in yet another example, 2 mL to 3 mL in yet another example, 2 mL to 4 mL in yet another example, 2 mL to 5 mL in yet another example, 2 mL to 6 mL in yet another example, 3 mL to 4 mL in yet another example, 3 mL to 5 mL in yet another example, and 3 mL to 6 mL in yet another example, for each injection site. The therapeutic composition in the injection volume can be administered using one or more units or sub-volumes. In an unlimiting example, a 6 mL injection volume can be administered using 2 injections each has a volume of 3 mL.

In some embodiments, the therapeutic composition is formulated to treat a CNS disorder comprising depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, or a combination thereof.

In some embodiments, the therapeutic composition is formulated to treat a CNS disorder selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

In some embodiments of the therapeutic composition, the neuroactive steroid (NAS) comprises brexanolone, long-acting brexanolone, slow-releasing brexanolone, ganaxolone, zuranolone, a prodrug thereof, and a combination thereof.

In some embodiments of the therapeutic composition, the neuroactive steroid (NAS) is selected from the group consisting of brexanolone, long-acting brexanolone, slow-releasing brexanolone, ganaxolone, zuranolone, a prodrug thereof, and a combination thereof.

In some embodiments, the therapeutic composition comprises any pharmaceutical composition disclosed herein.

In some embodiments, the neuroactive steroid (NAS) comprises a brexanolone crystalline form disclosed herein.

In some embodiments, The NAS comprises Brexanolone aqueous suspension BRII-296, a long-acting brexanolone.

In some embodiments, the therapeutic composition comprises the neuroactive steroid in a single NAS dose of about 50 mg to 1,000 mg of the neuroactive steroid per therapeutic packing unit. In some embodiments, the single NAS dose can be in a range of from about 50 mg to about 600 mg of the neuroactive steroid per therapeutic packing unit.

The therapeutic composition can be formulated for the neuroactive steroid to reach a maximum plasma concentration (Cₘₐₓ) in about 30 minutes to 6 hours and maintains a plasma concentration of more than about 5% of the Cₘₐₓ for at least about 5 days, in the subject, after a single dose of the neuroactive steroid.

In some embodiments of the therapeutic composition, the steroid composition comprises corticosteroid comprising alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate (such as Decadron-LA), dexamethasone sodium (such as Decadron or Solurex), fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate (such as Depo-Medrol), mometasone, mometasone furoate, prednicarbate, prednisolone, prednisone, tixocortol pivalate, triamcinolone acetonide, Triamcinolone hexacetonide (such as Aristospan), triamcinolone acetonide (such as Kenalog), or a combination thereof.

In some embodiments, the corticosteroid is selected from the group consisting of methylprednisolone acetate (such as Depo-Medrol), triamcinolone acetonide (such as Kenalog), triamcinolone hexacetonide (such as Aristospan), dexamethasone acetate (such as Decadron-LA), dexamethasone sodium (such as Decadron or Solurex), and a combination thereof.

In some embodiments, the therapeutic composition is formulated by pre-mixing the NAS and one or more of the corticosteroids prior to injection. In some embodiments, the therapeutic composition is formulated for prophylaxis treatment of injection site reaction caused or induced by the injection of a neuroactive steroid in a subject.

In some embodiments, the therapeutic composition is formulated by pre-mixing a long-acting brexanolone and one or more of the corticosteroids prior to injection. In some embodiments, the therapeutic composition is formulated by pre-mixing a long-acting brexanolone, such as BRII-296 with one or more of alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate (such as Decadron-LA), dexamethasone sodium (such as Decadron or Solurex), fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate (such as Depo-Medrol), mometasone, mometasone furoate, prednicarbate, prednisolone, prednisone, tixocortol pivalate, triamcinolone acetonide, triamcinolone hexacetonide (such as Aristospan), triamcinolone acetonide (such as Kenalog), or a combination thereof.

The therapeutic composition can be formulated to comprise the steroid composition in a single steroid dosage in a range of from about 10 mg to about 450 mg of the steroid composition per therapeutic packing unit. Each unit can be packaged in a vial, an ampoule, a syringe, a tube, or any packaging format that is suitable for injections.

In certain embodiments, the therapeutic composition is formulated to comprise the steroid composition in a range of from about 10 mg to about 450 mg of the steroid composition and about 50 mg to 1,000 mg of the neuroactive steroid per therapeutic packing unit.

In some embodiments, the therapeutic composition is formulated for treatment or prevention of postpartum depression (PPD) in a lactating woman who is breastfeeding.

The therapeutic compositions disclosed herein can include a steroid composition that comprises a steroid, e.g., a corticosteroid. In some embodiments, the corticosteroid is any of the corticosteroids disclosed herein. In some embodiments, the corticosteroid is selected from the group consisting of methylprednisolone, methylprednisolone acetate, cortisone, cortisone acetate, hydrocortisone, hydrocortisone aceponate, hydrocortisone acetate, hydrocortisone buteprate, hydrocortisone butyrate, hydrocortisone valerate, prednisolone, prednisone, and a combination thereof.

In some embodiments, the therapeutic composition is formulated for treating a central nervous system (CNS) disorder and preventing severe injection site reaction (ISR) in a subject. In some embodiments, the therapeutic composition comprises the steroid composition formulated in a single site steroid dosage in a range of from 10 to 100 mg of the steroid composition for each of the injection site and the neuroactive steroid (NAS) at a single site NAS dosage in a range of from 30 mg to 1200 mg of the neuroactive steroid for each of the injection site. Aforementioned dosage ranges and sub-ranges can be suitable.

In some embodiments, the therapeutic composition comprises the steroid composition and the neuroactive steroid in an injection volume in a range of from 0.5 mL to 6 mL for each of the injection site. Aforementioned volume ranges and sub-ranges can be suitable.

Unless expressly stated to the contrary, all ranges cited herein are inclusive.

Suitable for the pharmaceutical composition and the therapeutical composition of this invention, the excipient comprises surfactant, emulsifier, filler, carrier, isotonicifier, dispersing agent, viscosity modifier, resuspending agent, buffer or a combination thereof.

Examples of pharmaceutically acceptable excipients include a pharmaceutical surfactant, emulsifier, filler, carrier, isotonicifier, dispersing agent, viscosity modifier, resuspending agent, buffer or a combination thereof. Pharmaceutical excipients typically do not have properties of a medicinal or drug active ingredient, also known as active pharmaceutical ingredient (API) and are typically used to streamline the manufacture process or packaging of the active ingredients, or to deliver an API to a patient or other subject. Pharmaceutical acceptable carrier, excipients or inactive ingredients from the Inactive Ingredients Database available from US FDA (https://www.fda.gov/drugs/drug-approvals-and-databases/inactive-ingredients-database-download) can be suitable. Some of Generally Recognized As Safe (GRAS) food substances available form US FDA's GRAS Substances (SCOGS) Database (https://www.fda.gov/food/generally-recognized-safe-gras/gras-substances-scogs-database) can also be suitable.

In embodiments of the present disclosure, the pharmaceutical acceptable carrier comprises acacia, animal oils, benzyl alcohol, benzyl benzoate, calcium stearate, carbomers, cetostearyl alcohol, cetyl alcohol, cholesterol, cyclodextrins, dextrose, diethanolamine, emulsifying wax, ethylene glycol palmitostearate, glycerin, glycerin monostearate, glycerol stearate, glyceryl monooleate, glyceryl monostearate, hydrous, histidine, hydrochloric acid, hydroxypropyl cellulose, hydroxypropyl-β-cyclodextrin (HPBCD), hypromellose (hydroxypropyl methylcellulose (HPMC)), lanolin, lanolin alcohols, lecithin, medium-chain triglycerides, metallic soaps, methylcellulose, mineral oil, monobasic sodium phosphate, monoethanolamine, oleic acid, polyyethylene glycols (PEG 3350, PEG 4000, PEG 6000), polyoxyethylene-polyoxypropylene copolymer (poloxamer), polyoxyethylene alkyl ethers, polyoxyethylene castor oil, polyoxyethylene castor oil derivatives, polyoxyethylene sorbitan fatty acid esters, polyoxyethylene stearates, polysorbate, polyoxyethylene (20) sorbitan monolaurate (Tween 20, Polysorbate 20), polyoxyethylene (20) sorbitan monooleate (Tween 80, Polysorbate 80), povidone, propylene glycol alginate, saline, sodium chloride, sodium citrate, sodium citrate dihydrate, sodium hydroxide, sodium lauryl sulfate, sodium phosphate monobasic, sodium phosphate dibasic, sorbitan esters, stearic acid, stearyl alcohol, sunflower oil, tragacanth, triethanolamine, vegetable oils, water, xanthan gum, or a combinations thereof.

In further embodiments, the pharmaceutical acceptable carrier comprises dextrose, glycerin, histidine, hydrochloric acid, hydroxypropyl cellulose, hydroxypropyl-β-cyclodextrin (HPBCD), hypromellose (hydroxypropyl methylcellulose (HPMC)), polyoxyethylene (20) sorbitan monolaurate (Tween 20, Polysorbate 20), polyyethylene glycols (PEG 3350, PEG 4000, PEG 6000), polyoxyethylene-polyoxypropylene copolymer (Poloxamer 188, Poloxamer 407), polyoxyethylene (20) sorbitan monooleate (Tween 80, Polysorbate 80), saline, sodium chloride, sodium citrate, sodium citrate dihydrate, sodium lauryl sulfate, sodium phosphate monobasic, sodium phosphate dibasic, or a combination thereof.

Further exemplary CNS conditions related to GABA-modulation can include, but are not limited to, sleep disorders (e.g., insomnia), mood disorders (e.g., depression, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD))), schizophrenia spectrum disorders (e.g., schizophrenia, schizoaffective disorder), convulsive disorders (e.g., epilepsy (e.g., status epilepticus (SE)), seizures), disorders of memory and/or cognition (e.g., attention disorders (e.g., attention deficit hyperactivity disorder (ADHD)), dementia (e.g., Alzheimer's type dementia, Lewis body type dementia, vascular type dementia), movement disorders (e.g., Huntington's disease, Parkinson's disease), personality disorders (e.g., anti-social personality disorder, obsessive compulsive personality disorder), autism spectrum disorders (ASD) (e.g., autism, monogenetic causes of autism such as synaptophathy's, e.g., Rett syndrome, Fragile X syndrome, Angelman syndrome), pain (e.g., neuropathic pain, injury related pain syndromes, acute pain, chronic pain), traumatic brain injury (TBI), vascular diseases (e.g., stroke, ischemia, vascular malformations), substance abuse disorders and/or withdrawal syndromes (e.g., addition to opiates, cocaine, and/or alcohol), tinnitus or a combination thereof.

In the methods of treatment disclosed herein, the disease can include sleep disorders, insomnia, mood disorders, depression, dysthymic disorder, mild depression, bipolar disorder, anxiety disorders, generalized anxiety disorder (GAD), social anxiety disorder, stress, post-traumatic stress disorder (PTSD), compulsive disorders, obsessive compulsive disorder (OCD), schizophrenia spectrum disorders, schizophrenia, schizoaffective disorder, convulsive disorders, epilepsy, status epilepticus (SE), seizures, disorders of memory and/or cognition, attention disorders, attention deficit hyperactivity disorder (ADHD), dementia, Alzheimer's type dementia, Lewis body type dementia, vascular type dementia, movement disorders, Huntington's disease, Parkinson's disease, personality disorders, anti-social personality disorder, obsessive compulsive personality disorder, autism spectrum disorders (ASD), autism, monogenetic causes of autism, synaptophathy's, Rett syndrome, Fragile X syndrome, Angelman syndrome, neuropathic pain, injury related pain syndromes, acute pain, chronic pain, traumatic brain injury (TBI), vascular diseases, stroke, ischemia, vascular malformations, substance abuse disorders and/or withdrawal syndromes, addition to opiates, addition to cocaine, addition to alcohol, tinnitus, or a combination thereof.

In certain embodiments, the disease is selected from anxiety, massive depression disorder, postpartum disorder, Alzheimer disease, Parkinson disease, epilepsy, focal onset seizures, PCDH19 pediatric epilepsy, pediatric genetic epilepsies, CDKL5 Deficiency Disorder (CDD), catamenial epilepsy, infantile spasms, Fragile X syndrome, depression, postpartum depression, or premenstrual syndrome.

In certain embodiments, provided herein is a method for preventing or reducing an injection site reaction (ISR) in a patient receiving an intramuscular (IM) injection of a neuroactive steroid (NAS) comprising: (a) administering a NAS to a patient by IM injection to treat a central nervous system (CNS) disorder related to gamma-aminobutyric acid type A (GABA_{A}) receptor modulation; and (b) administering a corticosteroid to the patient by IM injection to prevent or reduce an ISR produced from step (a). In some embodiments, two IM injections are administered to the patient.

In some embodiments, the NAS is brexanolone. In certain embodiments, the NAS is administered to the patient in an aqueous formulation. In some embodiments, the aqueous formulation comprises a suspension of NAS particles. In certain embodiments, the aqueous formulation comprises a suspension of brexanolone particles. In certain embodiments, the brexanolone particles have a particle size distribution with a Dv50 between 1 μm to 6 μm. In certain embodiments, the brexanolone particles have a Dv50 of about 3 μm. In certain embodiments, the brexanolone particles have a Dv90 between 3 μm to 12 μm. In certain embodiments, the brexanolone particles have a Dv90 of about 6 μm.

In some embodiments, the NAS is administered in an aqueous formulation comprising a suspension of brexanolone particles and one or more excipients. In some embodiments, the one or more excipients comprise PEG 3350, polysorbate 80, mannitol, sodium citrate and/or citric acid.

In some embodiments, step (a) and step (b) are performed as separate IM injections. In some embodiments, step (a) and step (b) are performed simultaneously, for example, where the NAS and corticosteroid are in an admixture administered to the patient by one or more IM injections.

In certain embodiments, the NAS and the corticosteroid are administered to the patient as an admixture in a single IM injection. In some embodiments, the NAS and the corticosteroid are administered as an admixture in two IM injections to the patient. In some embodiments, the NAS and the corticosteroid are administered to the patient as an admixture in two IM injections, wherein each injection has a volume between about 2 mL to about 4 mL. In some embodiments, the NAS and the corticosteroid are administered to the patient as an admixture in two IM injections, wherein each injection has a volume of about 3 mL.

In certain embodiments, the corticosteroid is methylprednisolone or a salt thereof (e.g., methylprednisolone acetate).

In some embodiments, the NAS and corticosteroid are administered to the patient as an aqueous formulation comprising a suspension of crystalline brexanolone particles (e.g., brexanolone polymorph Form A) as the NAS and methylprednisolone or a salt thereof (e.g., methylprednisolone acetate) as the corticosteroid.

In certain embodiments, provided herein is a method for reducing a depressive symptom in a patient with PPD comprising administering an aqueous formulation comprising brexanolone particles and methylprednisolone or a salt thereof (e.g., methylprednisolone acetate) to the patient with PPD by intramuscular or subcutaneous injection. In some embodiments, two IM or SC injections are administered to the patient.

In certain embodiments, provided herein is a method for reducing a depressive symptom in a patient with MDD comprising administering an aqueous formulation comprising brexanolone particles and methylprednisolone or a salt thereof (e.g., methylprednisolone acetate) to the patient with MDD by intramuscular or subcutaneous injection. In some embodiments, two IM or SC injections are administered to the patient.

In some embodiments, the methods also treat or prevent ISR in a subject receiving the IM or SC injection.

In some embodiments of the methods provided herein, the amount of brexanolone administered to the patient is from about 300 mg to about 700 mg, and the amount of methylprednisolone acetate administered to the patient is from about 20 mg to about 100 mg. In some embodiments, the amount of brexanolone administered to the patient is about 400, 450, 460, 470, 480, 490, 500, 510, 520, 530, 540, 550, 560, 570, 580,590, 600, 620, or 640 mg, and the amount of methylprednisolone acetate administered to the patient is about 30, 40, 50, 60, 70, 80, or 90 mg. In some embodiments, the amount of brexanolone administered is from about 500 mg to about 600 mg, and the amount of methylprednisolone acetate administered to the patient is from about 40 mg to about 80 mg. In some embodiments, the amount of brexanolone administered to the patient is about 600 mg, and the amount of methylprednisolone acetate administered to the patient is about 40 mg. In some embodiments, the amount of brexanolone administered to the patient is about 600 mg, and the amount of methylprednisolone acetate administered to the patient is about 80 mg. The amounts of brexanolone and methylprednisolone acetate can, for example, be administered as a combination composition (e.g., an admixture), or, as another example, be administered separately. Moreover, in some embodiments where brexanolone and methylprednisolone acetate are administered as combination composition (e.g., an admixture) the combination can be administered as a single injection. In other embodiments, the amounts of brexanolone and methylprednisolone acetate in the combination is administered in two or more injections (e.g., in split doses) to the patient. In specific embodiments, the amounts of brexanolone and methylprednisolone acetate in the combination are administered in two injections to the patient, each comprising equal amounts of the brexanolone and methylprednisolone.

This disclosure is further directed to the use of the neuroactive steroid and a steroid composition for manufacturing a medicament for treating a disease, wherein the disease comprises CNS conditions related to GABA-modulation, sleep disorders, insomnia, mood disorders, depression, dysthymic disorder, mild depression, bipolar disorder, anxiety disorders, generalized anxiety disorder (GAD), social anxiety disorder, stress, post-traumatic stress disorder (PTSD), compulsive disorders, obsessive compulsive disorder (OCD), schizophrenia spectrum disorders, schizophrenia, schizoaffective disorder, convulsive disorders, epilepsy, status epilepticus (SE), seizures, disorders of memory and/or cognition, attention disorders, attention deficit hyperactivity disorder (ADHD), dementia, Alzheimer's type dementia, Lewis body type dementia, vascular type dementia, movement disorders, Huntington's disease, Parkinson's disease, personality disorders, anti-social personality disorder, obsessive compulsive personality disorder, autism spectrum disorders (ASD), autism, monogenetic causes of autism, synaptophathy's, Rett syndrome, Fragile X syndrome, Angelman syndrome, neuropathic pain, injury related pain syndromes, acute pain, chronic pain, traumatic brain injury (TBI), vascular diseases, stroke, ischemia, vascular malformations, substance abuse disorders and/or withdrawal syndromes, addition to opiates, addition to cocaine, addition to alcohol, tinnitus, or a combination thereof.

The neuroactive steroid and the steroid composition disclosed herein can be suitable. The therapeutic composition that comprises a neuroactive steroid (NAS) that is a positive modulator of gamma-aminobutyric acid type A (GABA_{A}) receptor, or a pharmaceutically acceptable salt thereof, a steroid composition, and one or more pharmaceutical acceptable excipients, as disclosed herein, can be suitable. All ranges and sub-ranges disclosed above and hereafter in this disclosure can be suitable.

This disclosure is further directed to a kit comprising a steroid composition that comprises a corticosteroid and a pharmaceutical composition, wherein the steroid composition and the pharmaceutical composition each is formulated for intravenous (IV) injection, intramuscular (IM) injection or subcutaneous injection (SC).

In some embodiments, the kit comprises at least one steroid packing unit comprising about 1 mg to about 450 mg of the corticosteroid and at least one packing unit of the pharmaceutical composition.

In some embodiments, the kit comprises at least two steroid packing units each comprising about 20 mg to about 40 mg of the corticosteroid. In certain embodiments, each of the steroid packing units comprises 20 mg to 40 mg of a corticosteroid selected from alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, Triamcinolone hexacetonide, triamcinolone acetonide, and a combination thereof.

In certain embodiments, the pharmaceutical composition is selected from a vaccine, a nucleic acid vaccine, an mRNA vaccine, an attenuated vaccine, a viral vector vaccine, an antibiotic, a biologic drug, an antibody drug, adalimumab, etanercept, ixekizumab, secukinumab, ustekinumab, a tumor necrosis factor-α (TNF-α) inhibitor etanercept, a chemotherapy drug, bleomycin, CABENUVA (cabotegravir and rilpivirine extended-release injectable suspension), cabotegravir, carboplatin, carmustine, cisplatin, cladribine, cacarbazine, daunorubicin, denileukin diftitox, dolasetrone, doxorubicin, doxorubicin liposome, EMGALITY (galcanezumab-gnlm), epirubicin, etoposide, fulvestrant, idarubicin, ifosfamide, interferon (IFN) beta, mechlorethamine, mitomycin, mitoxantrone, paclitaxel, pamidronate, pegfilgrastim, plicamycin, rilpivirine, Streptozocin, teniposide, thiotepa, vinblastine, vincristine, vinorelbine, a neuroactive steroid (NAS), and a combination thereof.

In certain embodiments, the pharmaceutical composition comprises a neuroactive steroid (NAS) selected from the group consisting of brexanolone, long-acting brexanolone, slow-releasing brexanolone, ganaxolone, zuranolone, a prodrug thereof, and a combination thereof.

In some embodiments, the kit comprises at least one steroid packing unit comprising about 1 mg to about 450 mg of the corticosteroid and at least one NAS packing unit comprising about 50 mg to 1,000 mg of a neuroactive steroid. In certain embodiments, the kit comprises at least one steroid packing unit comprising about 20 mg to about 40 mg of the corticosteroid and at least one NAS packing unit comprising about 300 mg to 800 mg of the neuroactive steroid. In certain embodiments, the kit comprises at least two steroid packing units each comprising about 20 mg to about 40 mg of the corticosteroid and at least one NAS packing unit comprising about 300 mg to 800 mg of the neuroactive steroid. The aforementioned corticosteroid and neuroactive steroid can be suitable. The aforementioned ranges of dosages for the corticosteroid and neuroactive steroid can be suitable.

In certain embodiments, the kit can further comprise an admixing device selected from a sterile vial, a sterile tube, a sterile syringe, a sterile bag, or a combination thereof, for admixing the steroid composition and the pharmaceutical composition. The steroid composition and the pharmaceutical composition can be admixed in the admixing device prior to administering to a subject via injection. In certain embodiments, two or more steroid packing units is admixed together with the pharmaceutical composition, such as the NAS packing unit comprising the neuroactive steroid.

### NUMBERED EMBODIMENTS OF THE DISCLOSURE

1. A process for treating or preventing injection site reaction (ISR) in a subject in need thereof, said process comprising administering to the subject via injection a corticosteroid composition and a pharmaceutical composition comprising a neuroactive steroid (NAS), wherein:

(i) said corticosteroid composition is co-formulated with said pharmaceutical composition;

(ii) said corticosteroid composition is admixed with said pharmaceutical composition;

(iii) said corticosteroid composition is co-administered with said pharmaceutical composition at a co-administration site at or adjacent to said injection site of said pharmaceutical composition,

or combination thereof,

wherein said pharmaceutical composition is administered to said subject via intravenous (IV) injection, intramuscular (IM) injection or subcutaneous injection (SC) at said injection site.

2. The process of embodiments 1, wherein the corticosteroid composition is a corticocorticosteroid composition.

3. The process of embodiments 1 or 2, wherein the corticosteroid composition and pharmaceutical composition are administered via injection at at least one injection site.

4. The process of embodiments 1 or 2, wherein said corticosteroid composition is admixed with said pharmaceutical composition prior to injection and administered together at said injection site.

5. The process of embodiments 1 or 2, wherein said corticosteroid composition is co-formulated with said pharmaceutical composition and administered together at said injection site.

6. The process of any one of embodiments 1-3, wherein said corticosteroid composition is co-administered to said subject 0 to 60 minutes from administration of said pharmaceutical composition to said subject.

7. The process of embodiments 5, wherein said corticosteroid composition is co-administered to said subject at said co-administration site that is 0 to 5 centimeters from said injection site.

8. The process of any one of embodiments 1-7, wherein said corticosteroid composition and/or said pharmaceutical composition is each administered to said subject in a therapeutic dose.

9. The process of any one of embodiments 1-7, wherein said corticosteroid composition and/or said pharmaceutical composition is administered to said subject in a sub-therapeutic dose.

10. The process of any one of embodiments 1-9, wherein said corticosteroid composition comprises a corticosteroid selected from the group consisting of alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, triamcinolone hexacetonide, triamcinolone acetonide, and a combination thereof.

11. The process of embodiments 10, wherein said corticosteroid is selected from the group consisting of methylprednisolone, methylprednisolone acetate, cortisone, cortisone acetate, hydrocortisone, hydrocortisone aceponate, hydrocortisone acetate, hydrocortisone buteprate, hydrocortisone butyrate, hydrocortisone valerate, prednisolone, prednisone, and a combination thereof.

12. The process of embodiments 10, wherein said corticosteroid is selected from the group consisting of methylprednisolone acetate, triamcinolone acetonide, triamcinolone hexacetonide, dexamethasone acetate, dexamethasone sodium, and a combination thereof.

13. The process of any one of embodiments 1-12, wherein said corticosteroid composition is administered to said subject in a single dose comprising from 0.01 to 50 mg of said steroid per kilogram of body weight of said subject.

14. The process of any one of embodiments 1-12, wherein said corticosteroid composition is administered to said subject in a single dose comprising from 0.01 to 2 mg of said steroid per kilogram of body weight of said subject.

15. The process of any one of embodiments 1-14, wherein said corticosteroid composition dose comprises from about 1 mg to about 450 mg of said steroid.

16. The process of any one of embodiments 1-8, wherein said corticosteroid composition is administered to said subject in a dose comprising from 10 to 100 mg of said steroid for each of said injection site and said pharmaceutical composition is administered to said subject in dose comprising from 30 mg to 1200 mg of said active agent.

17. The process of embodiments 16, wherein said corticosteroid composition and said pharmaceutical composition are formulated in a single injection volume ranging from 0.5 mL to 6 mL.

18. The process of any one of embodiments 1-17, wherein said ISR is caused or induced by said pharmaceutical composition comprising a neuroactive steroid (NAS).

19. The process of any one of embodiments 1-18, wherein said pharmaceutical composition comprising a neuroactive steroid is administered to said subject in a single dose comprising about 0.05 to 50 mg of said NAS per kilogram of body weight of said subject.

20. The process of embodiments 19, wherein said single dose comprises about 50 mg to 1,000 mg of said neuroactive steroid.

21. The process of embodiments 19, wherein said single dose comprises about 50 mg to about 600 mg of said neuroactive steroid.

22. The process of any one of embodiments 1-21, wherein said neuroactive steroid reaches a maximum plasma concentration (Cₘₐₓ) in about 30 minutes to 6 hours and maintains a plasma concentration of more than about 5% of said Cₘₐₓ for at least about 5 days, in said subject, after a single dose of said pharmaceutical composition.

23. The process of any one of embodiments 1-23, wherein said neuroactive steroid (NAS) is selected from the group consisting of brexanolone, long-acting brexanolone, slow-releasing brexanolone, ganaxolone, zuranolone, a prodrug thereof, and a combination thereof.

24. The process of any one of embodiments 1-23, wherein said neuroactive steroid (NAS) is selected from the group consisting of brexanolone, long-acting brexanolone, slow-releasing brexanolone, a prodrug thereof, and a combination thereof.

25. The process of any one of embodiments 1-24, wherein said pharmaceutical composition comprising a neuroactive steroid is formulated to treat a central nervous system (CNS) disorder related to GABA_{A} receptor modulation selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

26. The process of any one of embodiments 1-25, wherein said subject is a lactating woman who is breastfeeding and said disease is postpartum depression (PPD).

27. The process of any one of embodiments 1-26, wherein the process further comprises administering to said subject a therapeutically effective amount of one or more additional active agents.

28. A method for treating a central nervous system (CNS) disorder related to gamma-aminobutyric acid type A (GABA_{A}) receptor modulation in a subject in need thereof, said method comprising administering (i) a pharmaceutical composition comprising a neuroactive steroid (NAS) that is a positive modulator of the GABA_{A} receptor, or a pharmaceutically acceptable salt thereof, and (ii) a corticosteroid composition to said subject via injection, wherein said pharmaceutical composition and said corticosteroid composition are administered to said subject via intramuscular (IM) injection or subcutaneous injection (SC).

29. The method of embodiment 28, wherein the subject has or is susceptible to an injection site reaction.

30. The method of embodiment 29, wherein the ISR is severe ISR.

31. The method of any one of embodiments 28-30, wherein said pharmaceutical composition and said corticosteroid composition are administered to said subject via intramuscular (IM) injection or subcutaneous injection (SC) at at least one injection site.

32. The method of any one of embodiments 28-31, wherein (1) said corticosteroid composition is co-formulated with said pharmaceutical composition comprising a neuroactive steroid (NAS) and administered together to said subject via injection, (2) said corticosteroid composition is admixed with said neuroactive steroid (NAS) and administered together to said subject via injection, (3) said corticosteroid composition is co-administered with said neuroactive steroid (NAS), or a combination thereof.

33. The method of any one of embodiments 28-31, wherein said corticosteroid composition is admixed with said pharmaceutical composition comprising a neuroactive steroid (NAS) prior to injection and administered together at said injection site.

34. The method of any one of embodiments 28-31, wherein said corticosteroid composition is co-formulated with said pharmaceutical composition comprising a neuroactive steroid (NAS) and administered together at said injection site.

35. The method of any one of embodiments 28-31, wherein said corticosteroid composition is co-administered to said subject within 0 to 60 minutes before or after said neuroactive steroid (NAS) is administered to said subject.

36. The method of embodiment 35, wherein said corticosteroid composition is co-administered to said subject at least at a co-admin site that is within 0 to 5 centimeters from said injection site.

37. The method of any one of embodiments 28-36, wherein said neuroactive steroid (NAS) is selected from the group consisting of brexanolone, long-acting brexanolone, slow-releasing brexanolone, ganaxolone, zuranolone, a prodrug thereof, and a combination thereof.

38. The method of any one of embodiments 28-36, wherein said neuroactive steroid (NAS) is selected from the group consisting of brexanolone, long-acting brexanolone, slow-releasing brexanolone, a prodrug thereof, and a combination thereof.

39. The method of any one of embodiments 28-38, wherein of said pharmaceutical composition is administered to said subject in a single dose comprising about 0.01 to 50 mg of said neuroactive steroid per kilogram of body weight of said subject.

40. The method of embodiment 39, wherein said single dose comprises about 50 mg to 1,000 mg of said neuroactive steroid.

41. The method of embodiment 39, wherein said single dose comprises from about 50 mg to about 600 mg of said neuroactive steroid.

42. The method of any one of embodiments 28-38, wherein said corticosteroid composition is administered to said subject in a single dose comprising from 10 to 100 mg of said steroid and said pharmaceutical composition is administered to said subject in a single dose comprising from 30 mg to 1200 mg of said neuroactive steroid (NAS).

43. The method of embodiment 42, wherein said corticosteroid composition and said pharmaceutical composition comprising a neuroactive steroid are formulated in an injection volume ranging from 0.5 mL to 6 mL for each of said injection site.

44. The method of any one of embodiments 28-43, wherein said neuroactive steroid reaches a maximum plasma concentration (Cₘₐₓ) in about 30 minutes to 6 hours and maintains a plasma concentration of more than about 5% of said Cₘₐₓ for at least about 5 days, in said subject, after a single dose of said neuroactive steroid.

45. The method of any one of embodiments 28-44, wherein said corticosteroid composition comprises a corticosteroid selected from the group consisting of alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, triamcinolone hexacetonide, triamcinolone acetonide, and a combination thereof.

46. The method of embodiment 45, wherein said corticosteroid is selected from the group consisting of methylprednisolone acetate, triamcinolone acetonide, triamcinolone hexacetonide, dexamethasone acetate, dexamethasone sodium, and a combination thereof.

47. The method of any one of embodiments 28-46, wherein said subject is a lactating woman who is breastfeeding and said disease is postpartum depression (PPD).

48. The method of any one of embodiments 28-46, wherein said CNS disorder is selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

49. The method of any one of embodiments 28-46, wherein the CNS disorder is PPD.

50. A therapeutic composition comprising (i) a neuroactive steroid (NAS) that is a positive modulator of gamma-aminobutyric acid type A (GABA_{A}) receptor, or a pharmaceutically acceptable salt or composition thereof, (ii) a corticosteroid composition, and (iii) one or more pharmaceutical acceptable excipients.

51. The therapeutic composition of embodiment 50, wherein said therapeutic composition is formulated for intravenous (IV) injection, intramuscular (IM) injection or subcutaneous injection (SC).

52. The therapeutic composition of embodiment 50 or 51, wherein said therapeutic composition is formulated for treating a CNS disorder selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

53. The therapeutic composition of embodiment 52, wherein said CNS disorder is PPD.

54. The therapeutic composition of any one of embodiments 50-53, wherein said therapeutic composition is formulated to treat or prevent severe ISR caused or induced by IV, IM, or SC injection of the NAS.

55. The therapeutic composition of any one of embodiments 50-54, wherein said neuroactive steroid (NAS) is selected from the group consisting of brexanolone, long-acting brexanolone, slow-releasing brexanolone, ganaxolone, zuranolone, a prodrug thereof, and a combination thereof.

56. The therapeutic composition of any one of embodiments 50-55, wherein said therapeutic composition comprises from about 50 mg to 1,000 mg of said neuroactive steroid in a single dose.

57. The therapeutic composition of any one of embodiments 50-55, wherein said therapeutic composition comprises from about 50 mg to about 600 mg of said neuroactive steroid in a single dose.

58. The therapeutic composition of any one of embodiments 50-57, wherein said therapeutic composition is formulated for said neuroactive steroid to reach a maximum plasma concentration (Cₘₐₓ) in about 30 minutes to 6 hours and maintains a plasma concentration of more than about 5% of said Cₘₐₓ for at least about 5 days, in said subject, after a single dose of said neuroactive steroid (NAS).

59. The therapeutic composition of any one of embodiments 50-58, wherein said corticosteroid composition comprises a corticosteroid selected from the group consisting of alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, triamcinolone hexacetonide, triamcinolone acetonide, and a combination thereof.

60. The therapeutic composition of embodiment 59, wherein said corticosteroid is selected from the group consisting of methylprednisolone acetate, triamcinolone acetonide, triamcinolone hexacetonide, dexamethasone acetate, dexamethasone sodium, and a combination thereof.

61. The therapeutic composition of embodiment 59, wherein said corticosteroid is methylprednisolone acetate.

62. The therapeutic composition of any one of embodiments 50-55, wherein the corticosteroid composition comprises from about 10 mg to about 450 mg of said steroid.

63. The therapeutic composition of any one of embodiments 50-62, wherein said therapeutic composition is formulated for treatment or prevention of postpartum depression (PPD) in a lactating woman who is breastfeeding.

64. The therapeutic composition of any one of embodiments 50-63, wherein said therapeutic composition comprises 1) a corticosteroid composition comprising a single site dose ranging from 10 to 100 mg of said steroid for each of said injection site and 2) said neuroactive steroid (NAS) at a single site dose ranging from 30 mg to 1200 mg of said neuroactive steroid.

65. The therapeutic composition of embodiment 64, wherein said therapeutic composition comprises said corticosteroid composition and said neuroactive steroid in an injection volume ranging from 0.5 mL to 6 mL.

66. The therapeutic composition of any one of embodiment 62-65, wherein the composition is provided as a therapeutic packing unit.

67. A kit comprising a corticosteroid composition that comprises a corticosteroid and a pharmaceutical composition, wherein said corticosteroid composition and said pharmaceutical composition each is formulated for intravenous (IV) injection, intramuscular (IM) injection or subcutaneous injection (SC).

68. The kit of embodiment 67, wherein said kit comprises at least one steroid packing unit comprising about 1 mg to about 450 mg of said corticosteroid and at least one packing unit of said pharmaceutical composition comprising a neuroactive steroid (NAS) selected from the group consisting of brexanolone, long-acting brexanolone, slow-releasing brexanolone, ganaxolone, zuranolone, a prodrug thereof, and a combination thereof.

69. The kit of embodiment 67 or 68, wherein said kit comprises at least one said steroid packing units comprising about 20 mg to about 40 mg of said corticosteroid and at least one said NAS packing unit comprising about 300 mg to 800 mg of said neuroactive steroid.

70. The kit of any one of embodiments 67-69 further comprising an admixing device selected from a sterile vial, a sterile tube, a sterile syringe, a sterile bag, or a combination thereof, for admixing said corticosteroid composition and said pharmaceutical composition.

The instant disclosure now will be exemplified in the following non-limiting examples.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1: Brexanolone Suspensions

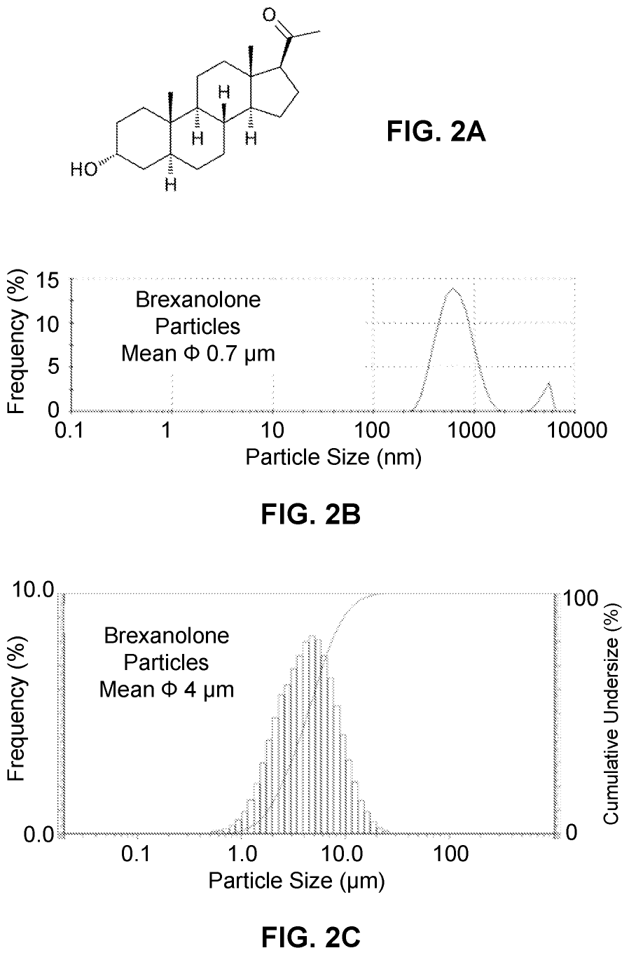

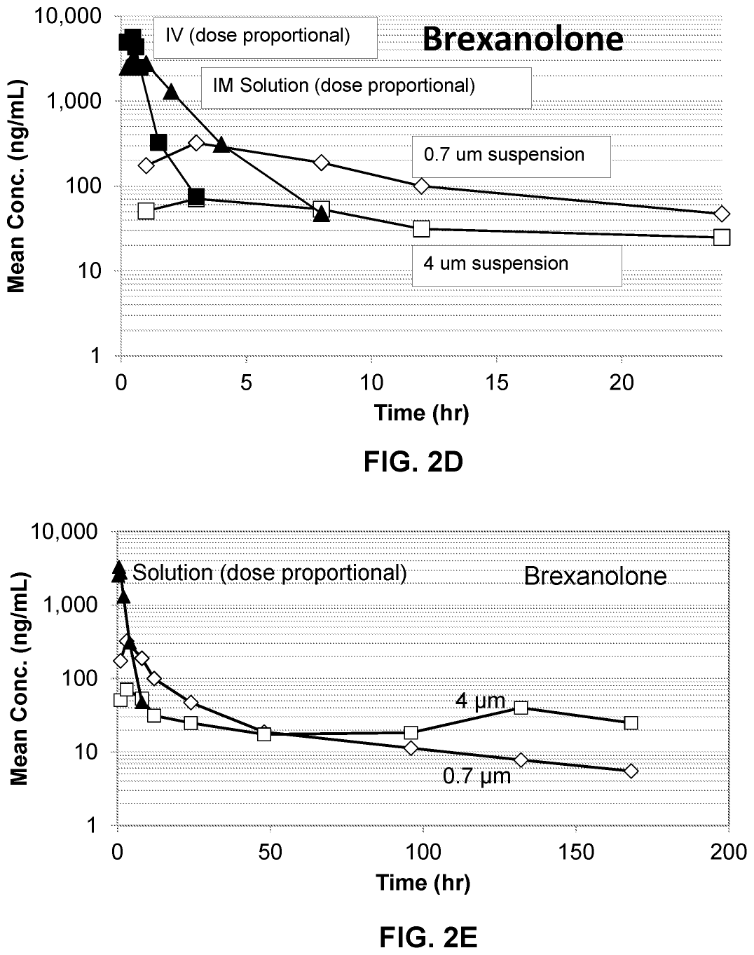

Brexanolone was purchased from commercial vender as an active pharmaceutical ingredient (API) (FIG. 2A). The article size of the commercial brexanolone is about 7 to 8 μm.

The commercial brexanolone was milled in the presence of water, saline, dextrose, HPMC, TWEEN 80, poloxamer 407 and glycerin with a rotation speed of about 300 to 500 rpm for about 20 to 30 minutes. The milling was performed for 1-5 cycles depending on the desired particle size. The milling media used was beads having a diameter of 0.1 to 1.0 mm.

By controlling the milling parameters, two sets of particles sizes were selected. One was small particle having a mean particle size of about 0.7 μm (FIG. 2B) and the other was large particle having a mean particle size of about 4.0 μm (FIG. 2C).

### Example 2: Pharmacokinetics (PKs) of Brexanolone Compositions in Rats

Suspensions of the 0.7 μm particles and the 4.0 μm particles were separately injected into rats via intramuscular (IM) injection at a dosage of 25 mg/kg of brexanolone. For comparison, comparative brexanolone solutions were injected via intramuscular (IM) injection at a dosage of 12.5 mg/kg, or intravenous (IV) injection at 1 mg/kg. Plasma brexanolone concentration were measured at indicated time points. Data are shown in FIG. 2D-FIG. 2E. PKs for the solutions were adjusted to have proportional doses.

### Example 3: Preparation of a Brexanolone Crystalline Form

Brexanolone was purchase from commercial vender and gently grounded to form slurries. The brexanolone samples were analyzed by following analytical techniques: FT-Raman spectroscopy, FT-IR spectroscopy, Differential calorimeter (DSC), Thermogravimetric analysis (TGA-IR), Polarized light microscopy (PLM) and Powder X-Ray diffraction (PXRD). The samples were determined to be white crystalline powder consisting of irregular particles with a wide range of sizes including large brittle chunks. The DSC analysis showed a melting endotherm at 174° C. ( [symbol] H=101 J/g). TGA analysis showed negligible (<0.1%) weight loss between 25-174° C., indicating that the brexanolone material is non-solvated.

Crystallizations were conducted in three modes: (1) temperature-cycled ripening of brexanolone slurries between 40-45° C. for two days (TC) (n=48); (2) heating the slurries to 40° C. followed by hot filtration, then storing the brexanolone solutions at 4° C. for up to two days (RC) (n=48); (3) evaporation of brexanolone solutions at ambient conditions for up to 7 days (EV) (n=48). A total of 48 solvent systems were screening for the crystal formation.

The brexanolone were crystallized from each of the solvents selected from the group consisting of dichloromethane (DCM), tetrahydrofuran (THF), ethyl acetate (EtOAc), dimethyl sulfoxide (DMSO), toluene, 2-propanol:water (9:1) (v/v), methanol (MeOH), 2-propanol (IPA), methyl t-butyl ether (MTBE), isopropyl ether (IPE), acetonitrile (MeCN), n-heptane, ethanol, water and a miscible combination thereof. All crystalline forms were analyzed using one or more of the analytical methods including PXRD, DSC, TGA and PLM.

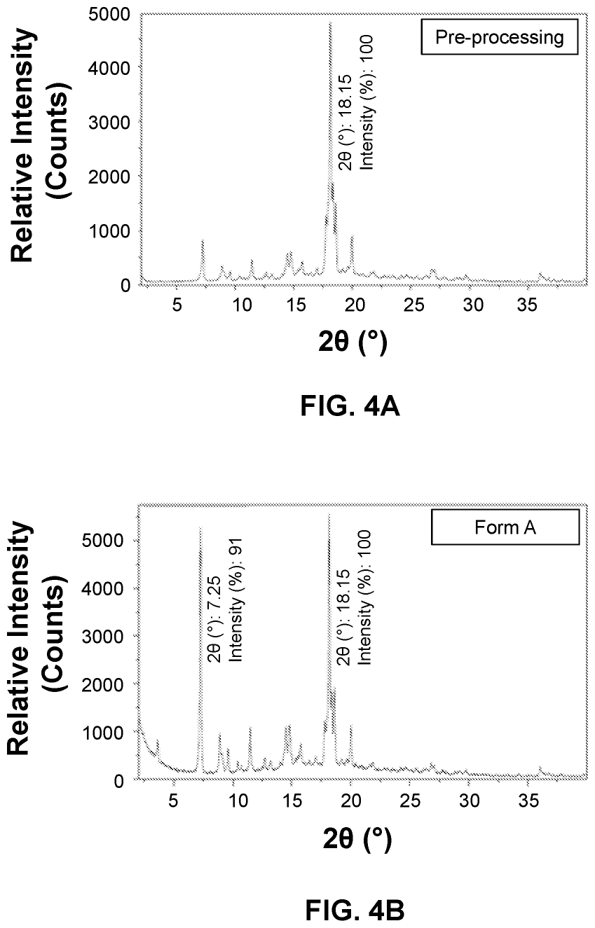

Brexanolone crystalline forms were characterized by 10 of the following peaks in Powder X-Ray Diffraction (PXRD) diffractograms, at 7.25, 8.88, 11.46, 14.50, 14.78, 17.77, 18.15, 18.32, 18.61 and 19.99±0.1 2θ)(°). A comparative commercial brexanolone exhibited a single peak at 18.15 (FIG. 4A). One brexanolone crystalline form, herein referred to as “polymorph Form A” exhibited characteristic two peaks at 7.25 and 18.15 2θ)(° having relative intensities of 91 and 100, respectively (FIG. 4B), were selected for use in the pharmaceutical composition in this disclosure. Relative intensities at the peaks 18.32, 18.61, 17.77, 14.78, 19.99, 14.50, 11.46 and 8.88 were 40%, 31%, 19%, 18%, 18%, 16%, 16% and 14%, respectively. The polymorph Form A brexanolone had particle sizes in a range of 1 μm to 100 μm that can be suitable for different formulations. The polymorph Form A was showing onset melting point of 174° C. ( [symbol] H=127 J/g). TGA analysis showed negligible (<0.1%) weight loss prior to the melting event indicating that the Form A is in a non-solvated form.

### Example 4: Manufacture of Brexanolone BRII-296 Formulation

The unit dose in Table 1 was prepared according to the following manufacturing process to produce brexanolone BRII-296 Formulation.

The manufacturing process for BRII-296 Suspension for Injection (herein “BRII-296”) comprises first preparing a formulation vehicle. The formulation vehicle was prepared by dissolving polysorbate 80, polyethylene glycol 3350 (PEG 3350), mannitol, citric acid monohydrate, and sodium citrate dihydrate in Water for Injection. The solution was filtered through one sterile 0.22 micron PVDF filter discarding first 200 mL of the filtrate. The pH of formulation vehicle was checked (target pH range is 6.0-7.0) and recorded. The required amount of brexanolone drug substance was weighed and added slowly to a container with required amount of formulation vehicle while mixing slowly to wet the drug substance completely. It was then homogenized for a minimum of 10 mins at 3000 RPM to obtain a uniform suspension with no aggregates present. The pre-mix suspension was transferred to a stainless still milling chamber containing clean and depyrogenated 1 mm yttrium-stabilized zirconium oxide grinding beads of a high density and hardness and milled until target particle size is achieved. In a non-limiting example, about 560 g of 1 mm beads were loaded into 250 mL milling chamber to which about 120 g of pre-mix suspension was added. Milling was conducted at 250 RPM for about 8-10 min with periodic checking of particle size (laser light diffraction). The milled suspension was transferred to a filling container after removing beads using a screen. While mixing the suspension slowly to maintain homogeneity, milled suspension was filled into pre-sterilized glass vials followed by stoppering and crimping the overseal to secure the stopper. During filling, fill weight was checked periodically as an in-process check.

Filled vials were terminally sterilized using e-beam irradiation with an external dose range of 38-42 kGy which corresponds to an internal dose range of 26-53 kGy.

All sterilized vials were inspected for defects and foreign particulates.

TABLE 1
Brexanolone aqueous suspension composition.

| Ingredient | Amount/vial (1.3 mL filled) |
|---|---|
| Brexanolone | 390 mg |
| PEG 3350 | 8.73 mg |
| Polysorbate 80 | 8.73 mg |
| Mannitol | 43.60 mg |
| Sodium citrate dihydrate | 2.23 mg |
| Citrate acid monohydrate | 0.14 mg |
| Water | QS |

### Example 5: Additional Injectable Brexanolone Pharmaceutical Compositions

A first brexanolone composition is produced using the suspension of the 0.7 μm particles prepared above to have a desired pharmacokinetics profile.

A second brexanolone composition is produced by mixing the suspensions of the 0.7 μm particles and 4.0 μm particles prepared above. The composition is adjusted to have about 10% of the 0.7 μm particles and about 90% of the 4.0 μm particles to have a desired pharmacokinetics profile.

A third brexanolone composition is produced by mixing the suspensions of the 0.7 μm particles and 4.0 μm particles prepared above. The composition is adjusted to have about 30% of the 0.7 μm particles and about 70% of the 4.0 μm particles to have a desired pharmacokinetics profile.

A fourth brexanolone composition is produced by mixing the suspensions of the 0.7 μm particles and 4.0 μm particles prepared above. The composition is adjusted to have about 40% of the 0.7 μm particles and about 60% of the 4.0 μm particles to have a desired pharmacokinetics profile.

A fifth brexanolone composition is produced by mixing the suspensions of the 0.7 μm particles and 4.0 μm particles prepared above. The composition is adjusted to have about 50% of the 0.7 μm particles and about 50% of the 4.0 μm particles to have a desired pharmacokinetics profile.

A sixth brexanolone composition is produced using the suspension of the 4 μm particles prepared above to have a desired pharmacokinetics profile.

### Example 6: Ganaxolone Suspensions

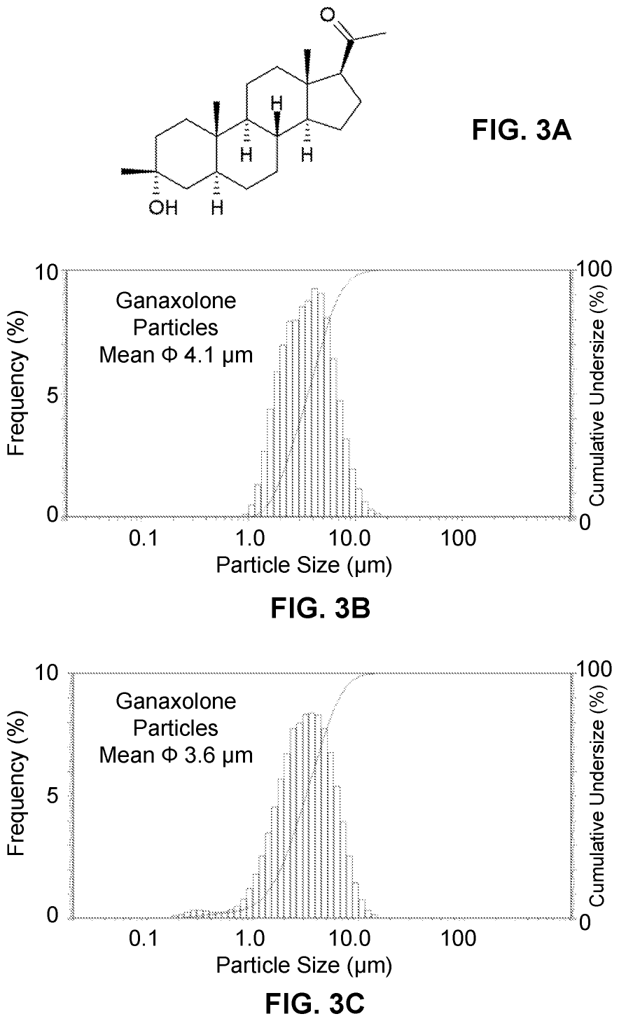

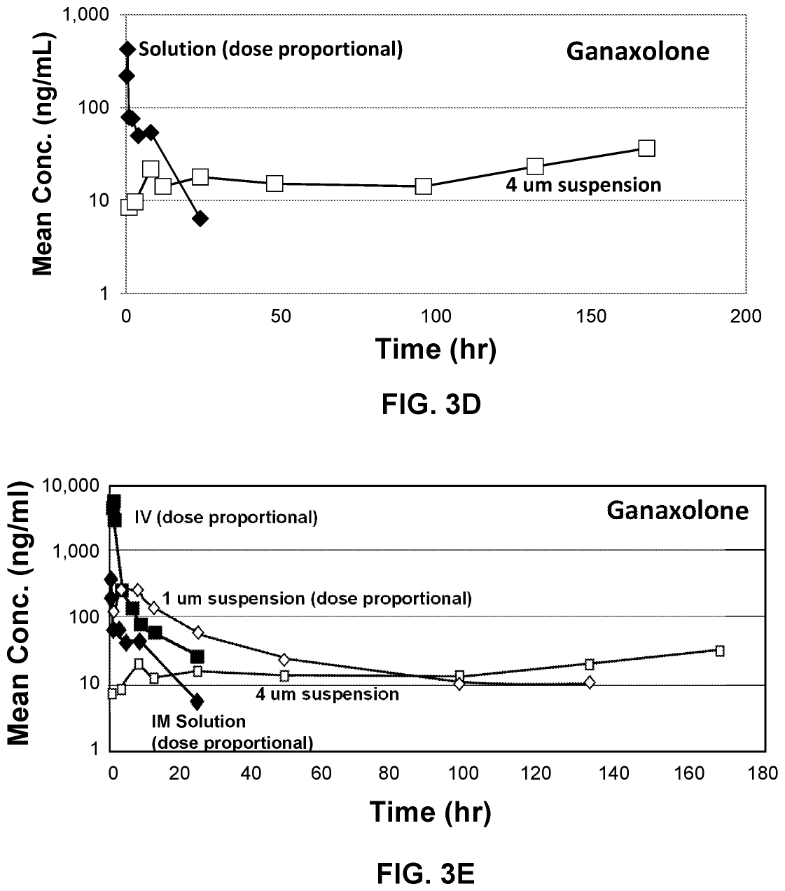

Ganaxolone was purchased from commercial vender as an active pharmaceutical ingredient (API) (FIG. 3A). The article size of the commercial brexanolone is about 47 μm.

The commercial ganaxolone was milled in the presence of water, saline, 1 mg/mL TWEEN 80 and 5 mg/mL HPMC with a rotation speed of about 200 rpm for about 20 minutes. The milling was performed for 3 cycles. The milling media used was beads having a diameter of 1.0 mm.

The milled particles had less than 1% of particles having sizes less than 1.5 μm with mean sizes of about 4.1 μm (FIG. 3B) about 3.6 μm (FIG. 3C) in two batches. Particles of having a mean particle size of about 1.0 μm were also produced.

### Example 7: Pharmacokinetics (PKs) of Ganaxolone Compositions in Rats

Suspensions of the particles having 1 μm and 4.1 μm were separately injected into rats via intramuscular (IM) injection at a dosage of 25 mg/kg of ganaxolone. For comparison, comparative ganaxolone solutions were injected via intramuscular (IM) injection at a dosage of 12.5 mg/kg, or intravenous (IV) injection at 1 mg/kg. Plasma brexanolone concentration were measured at indicated time points. Data are shown in FIG. 3D and FIG. 3E. PKs for the solutions were adjusted to have proportional doses.

### Example 8: Injectable Ganaxolone Pharmaceutical Compositions

A first ganaxolone composition is produced using the suspension of the 1 μm particles prepared above to have a desired pharmacokinetics profile.

A second ganaxolone composition is produced by mixing the suspensions of the 1 μm particles and 4.1 μm particles prepared above. The composition is adjusted to have about 10% of the 1 μm particles and about 90% of the 4.1 μm particles to have a desired pharmacokinetics profile.

A third ganaxolone composition is produced by mixing the suspensions of the 1 μm particles and 4.1 μm particles prepared above. The composition is adjusted to have about 30% of the 1 μm particles and about 70% of the 4.1 μm particles to have a desired pharmacokinetics profile.

A fourth ganaxolone composition is produced by mixing the suspensions of the 1 μm particles and 4.1 μm particles prepared above. The composition is adjusted to have about 40% of the 1 μm particles and about 60% of the 4.1 μm particles to have a desired pharmacokinetics profile.

A fifth ganaxolone composition is produced by mixing the suspensions of the 1 μm particles and 4.1 μm particles prepared above. The composition is adjusted to have about 50% of the 1 μm particles and about 50% of the 4.1 μm particles to have a desired pharmacokinetics profile.

A sixth ganaxolone composition is produced using the suspension of the 4.1 μm particles prepared above to have a desired pharmacokinetics profile.

### Examples 9: Unit Dose of Injectable Brexanolone Pharmaceutical Composition

A first unit dose of injectable brexanolone composition was packaged as 100 mg/mL of brexanolone injectable solution in a 1 mL vial.

A second unit dose of injectable brexanolone composition was packaged as 200 mg/mL of brexanolone injectable solution in a 1 mL vial.

A third unit dose of injectable brexanolone composition was packaged as 300 mg/mL of brexanolone injectable solution in a 1 mL vial.

A fourth unit dose of injectable brexanolone composition was packaged as 350 mg/mL of brexanolone injectable solution in a 1 mL vial.

A fifth unit dose of injectable brexanolone composition was packaged as 400 mg/mL of brexanolone injectable solution in a 1 mL vial.

A sixth unit dose of injectable brexanolone composition was packaged as 500 mg/mL of brexanolone injectable solution in a 1 mL vial.

A seventh unit dose of injectable brexanolone composition was packaged as 550 mg/mL of brexanolone injectable solution in a 1 mL vial.

An eighth unit dose of injectable brexanolone composition was packaged as 600 mg/mL of brexanolone injectable solution in a 1 mL vial.

### Example 10: Kenalog Injection and Brexanolone Plasma Concentrations

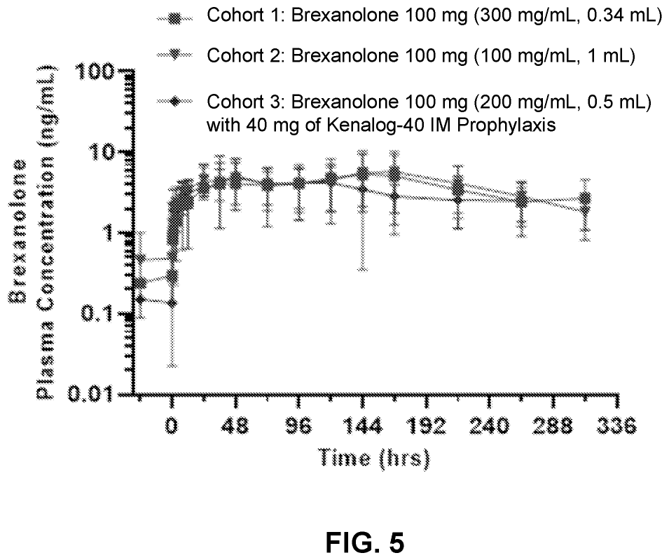

Healthy adults were administered 100 mg of BRII-296 at various concentrations and volumes via intramuscular (IM) injection (Cohort 1-3) (FIG. 5) under protocols approved by the Institutional Review Board (IRB). For IM steroid treatment (Cohort 3), each subject was injected 40 mg of Triamcinolone Kenalog-40 (40 mg/mL) (available from Bristol-Myers Squibb Company) via intramuscular (IM) injection 5-15 minutes after administration of BRII-296 injection. The Triamcinolone Kenalog-40 was injected within 1 cm of the BRII-296 injection site. The average body weight of the subjects was about 83 kg.

Plasma concentrations of brexanolone were measured at indicated time points. Data are shown in FIG. 5 demonstrating that there was no observed interference of the Kenalog co-injection to the absorption of brexanolone post BRII-296 IM injection indicating no observed drug-drug interaction (DDI) when brexanolone and Kenalog-40 were co-administered.

### Example 11: Kenalog Injection

Kenalog-10 is administered via intralesional injection to a patient in need for treating or preventing ISR. The Kenalog-10 contains 10 mg triamcinolone acetonide.

### Example 12: Aristospan Injection

A patient is administered via intramuscular (IM) brexanolone composition according to one of Examples above. Aristospan is injected near the brexanolone injection site using a sterile Aristospan 5 mg/mL suspension to treat or prevent ISR.

### Example 13: Depo-Medrol Injection

Depo-Medrol is administered via intramuscular (IM) injection to a patient in need for treating or preventing ISR using Depo-Medrol having concentrations of 20 mg/mL, 40 mg/mL, 80 mg/mL.

A patient is administered 80 mg of Depo-Medrol via intramuscular (IM) injection using 1 mL of an injection solution having 80 mg/mL Depo-Medrol.

Another patient is administered 40 mg of Depo-Medrol via intramuscular (IM) injection using 0.5 mL of an injection solution having 80 mg/mL Depo-Medrol.

Another patient is administered 20 mg of Depo-Medrol via intramuscular (IM) injection using 0.25 mL of an injection solution having 80 mg/mL Depo-Medrol.

Another patient is administered 40 mg of Depo-Medrol via intramuscular (IM) injection using 1 mL of an injection solution having 40 mg/mL Depo-Medrol.

Another patient is administered 20 mg of Depo-Medrol via intramuscular (IM) injection using 0.5 mL of an injection solution having 40 mg/mL Depo-Medrol.

Another patient is administered 20 mg of Depo-Medrol via intramuscular (IM) injection using 1 mL of an injection solution having 20 mg/mL Depo-Medrol.

Injection solution having higher concentrations, such as 80 mg/mL Depo-Medrol, is preferred.

### Example 14: Dexamethasone Injection

Dexamethasone sodium phosphate is administered via IV injection to a patient in need for treating or preventing ISR. The Dexamethasone sodium phosphate contains 4 mg (3.33 mg dexamethasone).

In another example, Dexamethasone sodium phosphate is administered via IM injection to a patient in need for treating or preventing ISR.

In another example, Dexamethasone sodium phosphate is administered via Intra-articular injection to a patient in need for treating or preventing ISR.

In another example, Dexamethasone sodium phosphate is administered via soft-tissue injection to a patient in need for treating or preventing ISR.

In another example, Dexamethasone sodium phosphate is administered via intralesional injection to a patient in need for treating or preventing ISR.

### Example 15: Betamethasone Injection

Betamethasone injectable suspension, available as Celestone® Soluspan® (available from Schering Corporation under respective registered trademarks), is administered via IM injection to a person in need for treating or preventing ISR at a lower dose of about 6 mg that is equivalent to 40 mg of Depo-Medrol because of Celestone Soluspan's intrinsic high potency.

### Example 16: Pre-mixed Injection Compositions

A first pre-mixed injection composition comprises BRII-296 and Kenalog-10.

A second pre-mixed injection composition comprises BRII-296 and Kenalog-40.

A third pre-mixed injection composition comprises BRII-296 and Aristospan.

A forth pre-mixed injection composition comprises BRII-296 and Depo-Medrol.

A fifth pre-mixed injection composition comprises BRII-296 and Dexamethasone.

A sixth pre-mixed injection composition comprises BRII-296 and Betamethasone.

A seventh pre-mixed injection composition comprises BRII-296 and alclometasone.

An eight pre-mixed injection composition comprises BRII-296 and dipropionate.

A ninth pre-mixed injection composition comprises BRII-296 and amcinonide.

A tenth pre-mixed injection composition comprises BRII-296 and Beclometasone.

An 11th pre-mixed injection composition comprises BRII-296 and betamethasone dipropionate.

A 12th pre-mixed injection composition comprises BRII-296 and betamethasone valerate.

A 13th pre-mixed injection composition comprises BRII-296 and budesonide.

A 14th pre-mixed injection composition comprises BRII-296 and ciclesonide.

A 15th pre-mixed injection composition comprises BRII-296 and clobetasol propionate.

A 16th pre-mixed injection composition comprises BRII-296 and clobetasone butyrate.

A 17th pre-mixed injection composition comprises BRII-296 and cortisone acetate.

A 19th pre-mixed injection composition comprises BRII-296 and clobetasol propionate.

A 20th pre-mixed injection composition comprises BRII-296 and desonide.

A 21st pre-mixed injection composition comprises BRII-296 and dexamethasone.

A 22nd pre-mixed injection composition comprises BRII-296 and dexamethasone acetate.

A 23rd pre-mixed injection composition comprises BRII-296 and dexamethasone sodium.

A 24th pre-mixed injection composition comprises BRII-296 and fluocinolone acetonide.

A 25th pre-mixed injection composition comprises BRII-296 and fluocinonide.

A 26th pre-mixed injection composition comprises BRII-296 and fluocortolone.

A 27th pre-mixed injection composition comprises BRII-296 and fluprednidene acetate.

A 28th pre-mixed injection composition comprises BRII-296 and halcinonide.

A 29th pre-mixed injection composition comprises BRII-296 and halometasone.

A 30th pre-mixed injection composition comprises BRII-296 and hydrocortisone.

A 31st pre-mixed injection composition comprises BRII-296 and hydrocortisone aceponate.

A 32nd pre-mixed injection composition comprises BRII-296 and hydrocortisone acetate.

A 33rd pre-mixed injection composition comprises BRII-296 and hydrocortisone buteprate.

A 34th pre-mixed injection composition comprises BRII-296 and hydrocortisone butyrate.

A 35th pre-mixed injection composition comprises BRII-296 and hydrocortisone valerate.

A 36th pre-mixed injection composition comprises BRII-296 and methylprednisolone.

A 37th pre-mixed injection composition comprises BRII-296 and methylprednisolone acetate.

A 38th pre-mixed injection composition comprises BRII-296 and mometasone.

A 39th pre-mixed injection composition comprises BRII-296 and mometasone furoate.

A 40th pre-mixed injection composition comprises BRII-296 and prednicarbate.

A 41st pre-mixed injection composition comprises BRII-296 and prednisolone.

A 42nd pre-mixed injection composition comprises BRII-296 and prednisone.

A 43rd pre-mixed injection composition comprises BRII-296 and tixocortol pivalate.

A 44th pre-mixed injection composition comprises BRII-296 and triamcinolone acetonide.

A 45th pre-mixed injection composition comprises BRII-296 and Triamcinolone hexacetonide.

A 46th pre-mixed injection composition comprises BRII-296 and triamcinolone acetonide.

### Injection Site Reaction (ISR) Assessment: Parameters and Categorization

The tolerability of an intramuscular (IM) injection of a pharmaceutical composition, e.g., a NAS, can be categorized, for example, as shown in Table 2.

TABLE 2
Categorization of Site Reactions to Intramuscular Injections^{†}

| | | | | Potentially Life |
|---|---|---|---|---|
| | Mild | Moderate | Severe | Threatening |
| Parameter | (Grade 1) | (Grade 2) | (Grade 3) | (Grade 4) |
| Injection Site | Pain or | Pain or | Pain or tenderness | Pain or tenderness |
| Pain or | tenderness | tenderness | causing inability | causing inability |
| Tenderness | causing no or | causing greater | to perform usual | to perform basic |
| Report only | minimal | than minimal | social and functional | self-care functions |
| one | limitation of | limitation of | activities | OR hospitalization |
| | use of limb | use of limb | | indicated |
| Injection Site | 2.5 to <5 cm in | ≥5 to <10 cm in | ≥10 cm in diameter | Potentially life- |
| Erythema or | diameter OR | diameter OR ≥25 | OR ≥100 cm² | threatening |
| Redness* | 6.25 to <25 cm² | to <100 cm² surface | surface area OR | consequences |
| Report only | surface area AND | area OR symptoms | ulceration OR | (e.g., abscess, |
| one | symptoms causing | causing greater | secondary infection | exfoliative |
| | no or minimal | than minimal | OR phlebitis OR | dermatitis, |
| | interference with | interference with | sterile abscess OR | necrosis involving |
| | usual social & | usual social & | drainage OR | dermis or deeper |
| | functional | functional | symptoms causing | tissue) |
| | activities | activities | inability to perform | |
| | | | usual social & | |
| | | | functional activities | |
| Injection Site | Same as for | Same as for | Same as for | Same as for |
| Induration or | injection site | injection site | injection site | injection site |
| Swelling | erythema or | erythema or | erythema or | erythema or |
| Report only | redness | redness | redness | redness |
| one | | | | |
| Injection Site | Itching localized | Itching beyond the | Generalized | Not applicable |
| Pruritis | to the injection | injection site that | itching causing | |
| | site that is relieved | is not generalized | inability to perform | |
| | spontaneously | OR itching localized | usual social & | |
| | or in <48 hours | to the injection | functional activities | |
| | of treatment | site requiring ≥48 | | |
| | | hours of treatment | | |

*Injection site erythema or redness should be evaluated and graded using the greatest single diameter or measured surface area.
^{†}Adapted from “Site Reactions to Injections and Infusions,” page 24 of US Department of Health and Human Services, Division of AIDS (DAIDS) Table for Grading the Severity of Adult and Pediatric Adverse Events, Corrected Version 2.1 (July 2017).

### Example 17: Pharmacokinetics of Brexanolone after Pre-mixed Injections

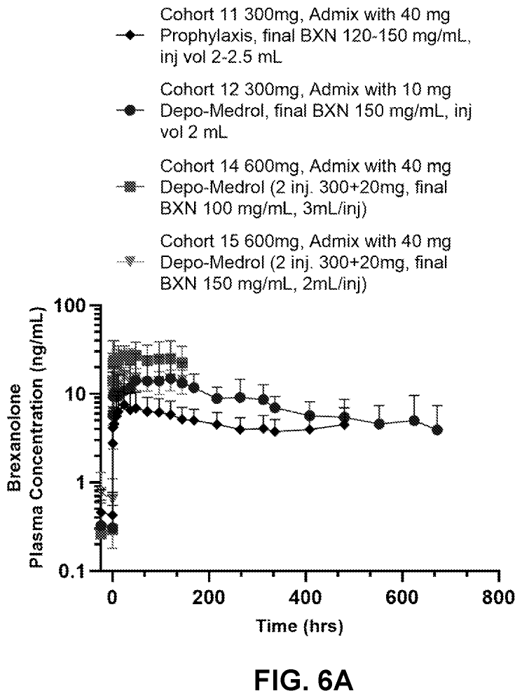

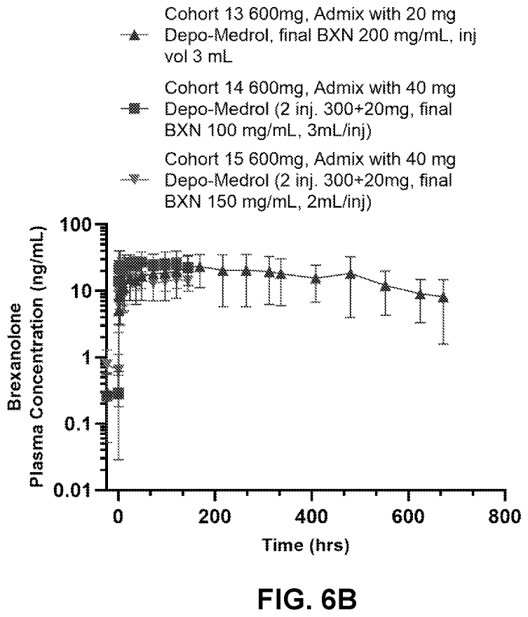

Plasma brexanolone (BXN) concentrations (pharmacokinetics, PK) after co-administration of BRII-296 and DEPO-MEDROL (methylprednisolone acetate) were determined in healthy male and female adults. Data are shown in FIG. 6A and FIG. 6B. ISR grades were assessed according to Table 2.

As shown in FIG. 6A:

Solid diamond: pre-mixed BRII-296 at 300 mg and Depo-Medrol 80 at 40 mg in an injection volume 2 to 2.5 mL with the final BXN concentration of about 120 to 150 mg/mL. Observed ISR was minimum. Data were from 8 subjects.

Solid circle: pre-mixed BRII-296 at 300 mg and Depo-Medrol 80 at 10 mg in an injection volume 2 mL with the final BXN concentration of about 150 mg/mL. Observed ISR was moderate to severe. Data were from 8 subjects.

Solid square: pre-mixed BRII-296 at 300 mg and Depo-Medrol 80 at 20 mg in an injection volume 3 mL with the final BXN concentration of about 100 mg/mL. Two injections were administered: 2×300 mg BXN/20 mg Depo-Medrol in 3 mL per injection. Observed ISR was moderate to severe. Data were from 8 subjects.

Solid triangle: pre-mixed BRII-296 at 300 mg and Depo-Medrol 80 at 20 mg in an injection volume 2 mL with the final BXN concentration of about 150 mg/mL. Two injections were administered: 2×300 mg BXN/20 mg Depo-Medrol in 2 mL per injection site. Observed ISR was moderate to severe. Data were from 4 subjects.

As shown in FIG. 6B:

Solid upward triangle: pre-mixed BRII-296 at 200 mg/mL and Depo-Medrol 80 at 20 mg in an injection volume 3 mL.

Solid square: pre-mixed BRII-296 at 300 mg/mL and Depo-Medrol 80 at 40 mg in an injection volume 3 mL with the final BXN concentration of about 100 mg/mL. Two injections were administered: 2×300 mg BXN/20 mg Depo-Medrol in 3 mL per injection site.

Solid downward triangle: pre-mixed BRII-296 at 300 mg/mL and Depo-Medrol 80 at 20 mg in an injection volume 2 mL with the final BXN concentration of about 150 mg/mL. Two injections were administered: 2×300 mg BXN/20 mg Depo-Medrol in 2 mL per injection.

### Example 18: ISR Data after Pre-mixed Injections

Healthy male and female adult subjects were injected with 300 mg or 600 mg of BRII-296, with (Cohort 10-16) or without (Cohort 4-5) Depo-Medrol (DM) according to approved protocols. Data are shown in Table 3. ISR grades were assessed according to Table 2. On per subject basis, 3 out of 8 subjects (37.5%) had Grade 3 ISRs at 300 mg without prophylaxis treatment. DM dose-dependent effects on ISR improvement were observed with 40 mg DM per injection provided the most optimal ISR profile (Table 3). N:number of subjects.

### Example 19: Methods of Treating or Preventing PPD and/or ISR

Eligible subjects will be administered 300 mg or 600 mg of BRII-296, with (Cohort 10-16) or without (Cohort 4-5) Depo-Medrol (DM) to evaluate the safety, tolerability, and/or efficacy of a BRII-296 with or without Depo-Medrol administered via intramuscular injection (IM) in adult women at risk of developing PPD.

Subjects with a Hamilton Depression Rating Scale (HAM-D)17 total score of 15 or higher on two occasions 1 week apart will be evaluated to confirm the presence of PPD and/or ISR. Percentage of subjects with an occurrence of a PPD episode and/or ISR during the 16-week treatment/prevention period for each cohort will be reported. DM dose-dependent effects on ISR improvement/prevention will also be observed and analyzed.

### Example 20: Kits Comprising the Steroid and NAS

A first kit comprises two vials each comprising 20 mg of Depo-Medrol and one vial comprising 300 mg of BRII-296.

A second kit comprises two vials each comprising 40 mg of Depo-Medrol and one vial comprising 300 mg of BRII-296.

A third kit comprises two vials each comprising 40 mg of Depo-Medrol and one vial comprising 600 mg of BRII-296.

A fourth kit comprises two vials each comprising 40 mg of Depo-Medrol, one vial comprising 20 mg of Depo-Medrol, and two vials each comprising 300 mg of BRII-296.

A fifth kit comprises two vials each comprising 40 mg of Depo-Medrol, one vial comprising 20 mg of Depo-Medrol, and one vial comprising 600 mg of BRII-296.

A sixth kit comprises three vials each comprising 20 mg of Depo-Medrol, and one vial comprising 600 mg of BRII-296.

TABLE 3
ISR data.

| | | Dosing | | # of ISRs Per Subjects | | | |
|---|---|---|---|---|---|---|---|
| Cohort | Dose (mg) | Volume (mL) | N | Total | Grade 1 | Grade 2 | Grade 3 |
| 4 | 300 | 1.0 | 4 | 4 | 4 | 1 | 1 |
| 5 | 300 | 3.0 | 4 | 4 | 1 | 4 | 2 |
| 10 | 300 + 40 mg DM Co-Inj | 1.5 | 6 | 5 | 1 | 5 | 0 |
| 11 | 300 mg + 40 mg DM Admix | 2.0 | 8 | 5 | 5 | 0 | 0 |
| 12 | 300 mg + 10 mg DM Admix | 2.0 | 8 | 3 | 0 | 3 | 1 |
| 14 | 600 2X(300 + 20 mg DM | 3.0 | 8 | 3 | 3 | 2 | 2 |
| | Admix) | | | | | | |
| 15 | 600 2X(300 + 20 mg DM | 2.0 | 4 | 4 | 3 | 2 | 3 |
| | Admix) | | | | | | |
| 16 | 600 2X(300 + 40 mg DM | 3.0 | 8 | 8 | 6 | 2 | 0 |
| | Admix) | | | | | | |

What is claimed is:

1. A therapeutic composition comprising (i) a neuroactive steroid (NAS) that is a positive modulator of gamma-aminobutyric acid type A (GABA_{A}) receptor, or a pharmaceutically acceptable salt or composition thereof, (ii) a corticosteroid composition, and (iii) one or more pharmaceutical acceptable excipients.

2. The therapeutic composition of claim 1, wherein said therapeutic composition is formulated for intravenous (IV) injection, intramuscular (IM) injection or subcutaneous injection (SC).

3. The therapeutic composition of claim 1, wherein said therapeutic composition is formulated for treating a CNS disorder selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

4. The therapeutic composition of claim 3, wherein said CNS disorder is PPD.

5. The therapeutic composition of claim 1, wherein said therapeutic composition is formulated to treat or prevent severe ISR caused or induced by IV, IM, or SC injection of the NAS.

6. The therapeutic composition of claim 1, wherein said neuroactive steroid (NAS) is selected from the group consisting of brexanolone, long-acting brexanolone, slow-releasing brexanolone, ganaxolone, zuranolone, a prodrug thereof, and a combination thereof.

7. The therapeutic composition of claim 1, wherein said therapeutic composition comprises from about 50 mg to 1,000 mg of said neuroactive steroid in a single dose.

8. The therapeutic composition of claim 1, wherein said therapeutic composition comprises from about 50 mg to about 600 mg of said neuroactive steroid in a single dose.

9. The therapeutic composition of claim 1, wherein said therapeutic composition is formulated for said neuroactive steroid to reach a maximum plasma concentration (Cₘₐₓ) in about 30 minutes to 6 hours and maintains a plasma concentration of more than about 5% of said Cₘₐₓ for at least about 5 days, in said subject, after a single dose of said neuroactive steroid (NAS).

10. The therapeutic composition of claim 1, wherein said corticosteroid composition comprises a corticosteroid selected from the group consisting of alclometasone dipropionate, amcinonide, beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, triamcinolone hexacetonide, triamcinolone acetonide, and a combination thereof.

11. The therapeutic composition of claim 10, wherein said corticosteroid is selected from the group consisting of methylprednisolone acetate, triamcinolone acetonide, triamcinolone hexacetonide, dexamethasone acetate, dexamethasone sodium, and a combination thereof.

12. The therapeutic composition of claim 10, wherein said corticosteroid is methylprednisolone acetate.

13. The therapeutic composition of claim 1, wherein the corticosteroid composition comprises from about 10 mg to about 450 mg of said steroid.

14. The therapeutic composition of claim 1, wherein said therapeutic composition is formulated for treatment or prevention of postpartum depression (PPD) in a lactating woman who is breastfeeding.

15. The therapeutic composition of claim 1, wherein said corticosteroid composition comprises a single site dose ranging from 10 to 100 mg of said steroid for each of said injection site, and said pharmaceutical composition comprises a single site dose ranging from 30 mg to 1200 mg of said neuroactive steroid.

16. The therapeutic composition of claim 15, wherein said therapeutic composition comprises said corticosteroid composition and said pharmaceutical composition in an injection volume ranging from 0.5 mL to 6 mL.

17. A method for treating a central nervous system (CNS) disorder related to gamma-aminobutyric acid type A (GABA_{A}) receptor modulation in a subject in need thereof, said method comprising administering (i) a pharmaceutical composition comprising a neuroactive steroid (NAS) that is a positive modulator of the GABA_{A} receptor, or a pharmaceutically acceptable salt thereof, and (ii) a corticosteroid composition to said subject via injection, wherein said pharmaceutical composition and said corticosteroid composition are administered to said subject via intramuscular (IM) injection or subcutaneous injection (SC).

18. The method of claim 17, wherein the subject has or is susceptible to an injection site reaction.

19. The method of claim 18, wherein the ISR is severe ISR.

20. The method of claim 17, wherein said pharmaceutical composition and said corticosteroid composition are administered to said subject via intramuscular (IM) injection or subcutaneous injection (SC) at at least one injection site.

21. The method of claim 17, wherein (1) said corticosteroid composition is co-formulated with said pharmaceutical composition comprising a neuroactive steroid (NAS) and administered together to said subject via injection, (2) said corticosteroid composition is admixed with said neuroactive steroid (NAS) and administered together to said subject via injection, (3) said corticosteroid composition is co-administered with said neuroactive steroid (NAS), or a combination thereof.

22. The method of claim 17, wherein said corticosteroid composition is admixed with said pharmaceutical composition comprising a neuroactive steroid (NAS) prior to injection and administered together at said injection site.

23. The method of claim 17, wherein said corticosteroid composition is co-formulated with said pharmaceutical composition comprising a neuroactive steroid (NAS) and administered together at said injection site.

24. The method of claim 17, wherein said corticosteroid composition is co-administered to said subject within 0 to 60 minutes before or after said neuroactive steroid (NAS) is administered to said subject.

25. The method of claim 24, wherein said corticosteroid composition is co-administered to said subject at least at a co-admin site that is within 0 to 5 centimeters from said injection site.

26. The method of claim 17, wherein said neuroactive steroid (NAS) is selected from the group consisting of brexanolone, long-acting brexanolone, slow-releasing brexanolone, ganaxolone, zuranolone, a prodrug thereof, and a combination thereof.

27. The method of claim 17, wherein said neuroactive steroid (NAS) is selected from the group consisting of brexanolone, long-acting brexanolone, slow-releasing brexanolone, a prodrug thereof, and a combination thereof.

28. The method of claim 17, wherein of said pharmaceutical composition is administered to said subject in a single dose comprising about 0.01 to 50 mg of said neuroactive steroid per kilogram of body weight of said subject.

29. The method of claim 28, wherein said single dose comprises about 50 mg to 1,000 mg of said neuroactive steroid.

30. The method of claim 28, wherein said single dose comprises from about 50 mg to about 600 mg of said neuroactive steroid.

31. The method of claim 17, wherein said corticosteroid composition is administered to said subject in a single dose comprising from 10 to 100 mg of said steroid and said pharmaceutical composition is administered to said subject in a single dose comprising from 30 mg to 1200 mg of said neuroactive steroid (NAS).

32. The method of claim 31, wherein said corticosteroid composition and said pharmaceutical composition comprising a neuroactive steroid are formulated in an injection volume ranging from 0.5 mL to 6 mL for each of said injection site.

33. The method of claim 17, wherein said neuroactive steroid reaches a maximum plasma concentration (Cₘₐₓ) in about 30 minutes to 6 hours and maintains a plasma concentration of more than about 5% of said Cₘₐₓ for at least about 5 days, in said subject, after a single dose of said neuroactive steroid.

34. The method of claim 17, wherein said corticosteroid composition comprises a corticosteroid selected from the group consisting of alclometasone dipropionate, amcinonide, Beclometasone, betamethasone, betamethasone dipropionate, betamethasone valerate, budesonide, ciclesonide, clobetasol propionate, clobetasone butyrate, cortisone, cortisone acetate, desonide, dexamethasone, dexamethasone acetate, dexamethasone sodium, fluocinolone acetonide, fluocinonide, fluocortolone, fluprednidene acetate, halcinonide, halometasone, hydrocortisone, hydrocortisone aceponate, hydrocortisone acetate, hydrocortisone buteprate, hydrocortisone butyrate, hydrocortisone valerate, methylprednisolone, methylprednisolone acetate, mometasone, mometasone furoate, prednicarbate, prednisolone, prednisone, tixocortol pivalate, triamcinolone acetonide, triamcinolone hexacetonide, triamcinolone acetonide, and a combination thereof.

35. The method of claim 34, wherein said corticosteroid is selected from the group consisting of methylprednisolone acetate, triamcinolone acetonide, triamcinolone hexacetonide, dexamethasone acetate, dexamethasone sodium, and a combination thereof.

36. The method of claim 17, wherein said subject is a lactating woman who is breastfeeding and said disease is postpartum depression (PPD).

37. The method of claim 17, wherein said CNS disorder is selected from the group consisting of depression, bipolar disorder, dementia, Huntington's disease, Parkinson's disease, traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), cyclin-dependent kinase like 5 (CDKL5) deficiency disorder, postpartum depression (PPD), treatment resistant depression (TRD), refractory status epilepticus (RSE), major depressive disorder (MDD), premenstrual dysphoric disorder (PMDD), persistent depressive disorder (PDD), seasonal affective disorder (SAD), secondary depression, post-finasteride syndrome, alcohol craving, smoking cessation, and a combination thereof.

38. The method of claim 37, wherein the CNS disorder is PPD.
